SEQUENCE LISTING (8 sequences)

SEQ ID NO: 1 - DNA, 26 bp, Artificial Sequence
  oligonucleotide primer with homology to adenovirus plasmid
aattgtttaaacggccctcgagccgt

SEQ ID NO: 2 - DNA, 25 bp, Artificial Sequence
  oligonucleotide primer with homology to adenovirus plasmid
atacggcctcgagggccgtttaaac

SEQ ID NO: 3 - DNA, 27 bp, Artificial Sequence
  oligonucleotide primer with homology to adenovirus plasmid
aattgtttaaacggccctcgaggccgt

SEQ ID NO: 4 - DNA, 25 bp, Artificial Sequence
  oligonucleotide primer with homology to adenovirus plasmid
atacggcctcgagggccgtttaaac

SEQ ID NO: 5 - DNA, 27 bp, Artificial Sequence
  oligonucleotide primer with homology to adenovirus plasmid
aattgtttaaacggccctcgaggccgt

SEQ ID NO: 6 - DNA, 25 bp, Artificial Sequence
  oligonucleotide primer with homology to adenovirus plasmid
atacggcctcgagggccgtttaaac

SEQ ID NO: 7 - DNA, 20 bp, Artificial Sequence
  oligonucleotide primer with homology to adenovirus plasmid
accgtcagatcgcctggaga

SEQ ID NO: 8 - DNA, 20 bp, Artificial Sequence
  oligonucleotide primer with homology to adenovirus plasmid
cgctgctaacgctgcaagag

(19) United States
(12) Patent Application Publication
(10) Pub. No.: US 2005/0123898 A1
(43) Pub. Date: Jun. 9, 2005

(54) System for producing clonal or complex populations of recombinant adenoviruses, and the application of the same

(76) Inventor: Moritz Hillgenberg, Berlin (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/483,962

(22) PCT Filed: Jul. 18, 2002
(86) PCT No.: PCT/EP02/08024

(30) Foreign Application Priority Data
Jul. 18, 2001 (EP) 01117379.6

(51) Int. Cl. C12Q 1/70; C12N 7/00; C12N 15/861

(52) U.S. Cl. 435/5; 435/456; 435/235.1

(57) ABSTRACT

The invention relates to a novel system for producing recombinant adenoviruses (rAd). The areas of application of said system are medicine, veterinary medicine, biotechnology, genetic engineering, and functional genomic analysis. The inventive system for producing rAds preferably consists of a donor virus, the packaging signal of which is (i) partially deleted and (ii) is surrounded by parallel recognition cites for a site-specific recombinase; a packaging cell line which expresses the site-specific recombinase; and donor plasmids containing (i) at least one recognition site for the site-specific recombinase, (ii) the full viral packaging signal, (iii) optionally two recognition sites for a rarely cutting restriction endonuclease, and (iv) insertion sites for foreign DNA or inserted foreign DNA.

The invention concerns a novel system for the generation of recombinant adenoviruses (rAd); Areas of application are medicine, veterinary science, biotechnology, gene technology and functional genome analysis.

The transfer of genes into cells is relevant for several reasons. The expression of genes introduced into cell culture systems enables e.g. the functional characterization of the coded proteins or its production. Furthermore, the transfer of therapeutically effective genes represents a new method for the treatment of human desease (gene therapy). As well as this, a great number of approaches are examined, with humans and livestock, through the transfer of immuno-stimulating and/or pathogenic-specific genes to achieve medically or veterinary effective immunization (vaccination). Finally, a special interest also exists in the field of the functional genome analysis with regard to efficient systems for gene transfer into cell-based functional test systems. Here, the vector system must also offer the possibility of the construction of complex gene libraries, as well as efficient gene transfer.

In the past years, numerous vectors have been developed for gene transfer in cells. Particularly with recombinant viral vectors, which are derived from retroviruses, adeno-associated viruses or adenoviruses, an efficient gene transfer in cells is possible (overview at: Verma, M. I. and Somia, N. (1997) Nature 389, 239-242). The so-called E1-deleted adenoviral vectors of the first generation were investigated intensively over the past decade as gene transfer vectors (Overview at: Bramson, J. L. et al. (1995). Curr. Op. Biotech. 6, 590-595). They are derived from the human adenovirus of the serotype 5 and are deleted in the essential E1 region, often also in the non-essential E3 region, through which up to 8 kb of foreign DNA can be inserted into the virus genome. These vectors can be produced to high titers on cells complementing the E1 deficiency. Due to their high level of stability, they can be well purified and stored. Recombinant adenoviruses have a broad spectrum of efficiently infected cell types in vitro and also allow an efficient gene transfer into different tissues in vivo. Clonal rAd populations are already used for many purposes for gene transfer in vitro and in vivo. Also, the employment of complex populations of rAd in the functional genome analysis—for example of cDNA expression libraries in the adenoviral context—appears very promising. With the previous methods of rAd generation, however, the generation of mixed rAd populations with a sufficient complexity is not possible.

A great number of methods have been described for rAd construction. The currently most usual methods are based on the insertion of the foreign DNA in the context of the adenovirus genome through homologous recombination. Two so-called shuttle plasmids are used in this case. A small shuttle plasmid contains the part of the adenovirus genome which should be manipulated. After the insertion of the foreign DNA into the smaller shuttle plasmid, the insertion in the context of the adenovirus genome is done through recombination with the larger shuttle plasmid, which provides the rest of the adenovirus genome. This recombination of the two shuttle plasmids can be done after co-transfection in 293 cells (McGrory, W. J., Bautista, D. S; and Graham, F. L. (1988) Virology 614-617) or after linearization and co-transformation in a recombination-competent E. coli strain (Chartier, C., Degryse, E., Gantzer, M., Dieterle, A., Pavirani, A. and Mehtali, M. (1996) J. Virol. 70: 4805-4810). Both methods are relatively labor-extensive due to system-inherent limitations: With the recombination in 293 cells, the problem exists that unrequired recombinant or wild type viruses can arise. For this reason, a clonal separation of the recombinant viruses is necessary through plaque assay on 293 cells and a thorough analysis of the separated rAd before the amplification. With the recombination in E. coli the problem exists that the recombination-competent bacterial strain supplies very low plasmid yields, through which the analysis of the recombined plasmids is complicated, since the retransformation of an E. coli strain is required with higher plasmid yield.

Newer methods for rAd construction, which have had little distribution up to now, are based on the insertion of foreign DNA into the context of the adenovirus genome through direct ligation. One method is based on the ligation of a fragment of the (manipulated) viral 5′-end with a fragment which contains the rest of the viral genome, followed by a transfection of the ligation products into 293 cells (Mizuguchi, H. and Kay, M. A. (1998) Hum. Gene Ther. 9: 2577-2583). Another method is based on the employment of the cosmid cloning technology. Cosmid vectors are used in this case, which contain the E1-deleted adenovirus genome and a polylinker with unique restriction sites for the insertion of foreign DNA. The ligation products from linearized cosmid vectors and foreign DNA to be inserted are packed in vitro into lambda phage heads. After infection by E. coli circular cosmids arise, from which linear rAd genomes can be set free by restriction digestion, which are then transfected into 293 cells (Fu, S and Deisseroth, A. B. (1997) Hum. Gene Ther. 8: 1321-1330).

The described previous methods for rAd generation have a feature in common, in that the infectious rAd arise from cloned DNA in 293 cells, where the cloned vector genome is either present linearly with terminal inverted terminal repeats (ITR's) or is present in the circular plasmid with a head-to-tail configuration of the ITR's. This cloned vector genome is distinguished structurally from natural adenovirus genomes, which contain a covalent linked viral protein (terminal protein, TP) at both ITR's. This is a result of a special feature of the adenoviral replication mechanism, with which the viral pre-terminal protein (pTP) serves as primer for the DNA synthesis and remains connected with the newly synthesized DNA on completion of the replication. Through a protease, the pTP is then processed to TP which, in the next replication round (as well as the ITR's) is an important part of the substrate which is identified from the replication machinery. Viral genomes without TP are identified 1000× worse, for instance, than naturally replicated viral genomes with TP (Overview in: Hay, R. T., Freeman, A., Leith, I., Monoghan, A. and Webster, A. (1995) Curr. Top. Microbial. immunol. 199: 31-48). The first replication of a cloned rAd vector genome without TP is thus a rare event (approx. 10-100 events per 10⁶ transfected 293 cells). For this reason, the above described methods are suitable only to obtain clonal populations of rAd. However, they are not suitable for the generation of complex populations of rAd, which would require an efficient conversion of a complex mixture of cloned vector genomes in a complex mixture of replicated rAd.

In the publication Hardy, S., Kitamura, M., Harris-Stansil, T., Dai, Y. and Phipps, L. M. (1997) J. Virol. 71, 1842-1849, a system is described for the generation of (clonal) populations of recombinant adenoviruses (rAd) through Cre/loxP recombination, comprising
Donor virus with complete packaging signal, which is framed by loxP recognition sequence,
Packaging cell line which expresses Cre,
Donor plasmid with 5′ITR, complete packaging signal, foreign DNA and a single loxP recognition sequence and two recognition sites for a restriction endonuclease (8 bp identification sequence).

A significant difference of the present invention in comparison to the system of Hardy, which is essential for the function of the system, is the partially deleted packaging signal in the donor virus, which enables the selection against the donor virus and is also required for the recombinants on normal 293 cells.

Pure preparations of rAd could be achieved by Hardy et al. (1997) only by co-transfection of virus DNA, together with the donor plasmid. For this, deproteined viral DNA, which thus does not have any terminal protein (TP), was used. The introduced donor virus substrate is thus distinguished from the infectious donor virus genomes with TP, which are introduced through infection in the case of the present invention. That there are such natural substrates for adenoviral replication is a significant advantage of the present invention. The introduction of the donor virus genome through infection was indeed also investigated by Hardy et al. (1997), however, the contamination with donor viruses was so high in the first amplification round, that this was not examined any further. The difference to the high purity of the invention is based on the placing at a disadvantage of the donor viruses due to the deleted packaging signal. The construction complex Ad populations were neither examined nor discussed by Hardy et al.

One task of the invention was therefore that of providing a system for the simple generation of a clonal recombinant adenovirus population. A further task was to provide a system with which complex recombinant adenoviruses can also be generated.

This task is solved invention-related through a system for the generation of recombinant adenoviruses, comprising
(a) At least one donor virus with one partially deleted viral packaging signal at least, which is framed by two recognition sites for a site-specific recombinase,
(b) A packaging cell line, which expresses the site-specific recombinase and
(c) At least one donor plasmid, which contains one or two recognition sites for the site-specific recombinase, the complete viral packaging signal and insertion sites for foreign DNA and/or inserted foreign DNA.

The invention-related novel method for rAd generation has decisive advantages compared the methods described up to now. On the one hand, the construction of clonal rAd populations is more rapid and less labor-extensive. On the other hand, complex mixed rAd populations can be generated, which was not possible with the previous state of the art. This creates for the first time the prerequisites for the construction of gene libraries in the adenoviral context. The significant feature of the invention-related new system is that the necessity for the conversion of cloned vector genome into infectious replicated vector genomes is bypassed, where the rAd are generated directly by enzymatic site-specific insertion of foreign DNA into a replicating virus. In this case, a site-specific recombinase is to be used, for example recombinases of the Int-Familie, such as Cre-recombinase or Flp-recombinase. The reactions catalyzed from these recombinases depend on the topology of the recognition sites: If two recognition sites lie in parallel-orientation on the same DNA molecule, then these site-specific recombinases catalyze the excision of the area in between as a circular molecule, where, at the excision point, a single recognition site remains. This reaction is reversible, however, the equilibrium, for thermodynamic reasons, is on the excision side (excision/insertion reaction). If two recognition sites are on different linear molecules, the site-specific recombinases catalyze the crosswise exchange of the terminals (terminal exchange). In this case also, an equilibrium reaction is involved, however, the equilibrium lies in the middle here, since the forward and back reactions are thermodynamically equivalent.

Furthermore, partially deleted and complete adenoviral packaging signals are used. Adenoviral packaging signals (Ψ) contain repeated sequence motives acting functionally additive, to which cellular factors, still not precisely characterized up to now, bind. The binding of these factors is necessary for an efficient packaging of the replicated viral genomes into the viral capsids. The packaging signal of the human adenovirus serotype 5 is currently best characterized: If individual or several of the repeated, functionally-additive-acting, sequence motives (“A repeats”) are deleted in the packaging signal, then the partially deleted packaging signal (ΔΨ), obtained in this way, causes a reduced packaging efficiency and thus a reduced virus growth (Schmid, S. I. and Hearing, P. (1997) J. Virol. 71: 3375-3384). The cellular factors furthermore represent a limiting substrate, so that, with simultaneous presence of a virus with a complete packaging signal, the growth reduction of a virus with a partially deleted packaging signal is additionally reinforced (Imler, J. L., Bout, A., Dreyer, D., Diederle, A., Schultz, H., Valerio, Dth, Mehtali, Mth and Pavirani, A. (1995) Hum. Gene Ther. 6: 711-721).

The employment of the novel method for rAd generation requires three significant components, which are part of the invention:
A donor virus, whose packaging signal (i) is partially deleted and (ii) is framed by parallel-oriented recognition sites for a site-specific recombinase.
A packaging cell line which expresses the site-specific recombinase.
A donor plasmid, which (i) contains one or two recognition sites for the site-specific recombinase, (ii) the complete viral packaging signal, (iii) where appropriate two recognition sites for a rare-cutting restriction endonuclease (in particular a restriction endonuclease with an identification sequence >8 bp, preferred >10 bp) and (iv) insertion sites for foreign DNA and/or inserted foreign DNA.

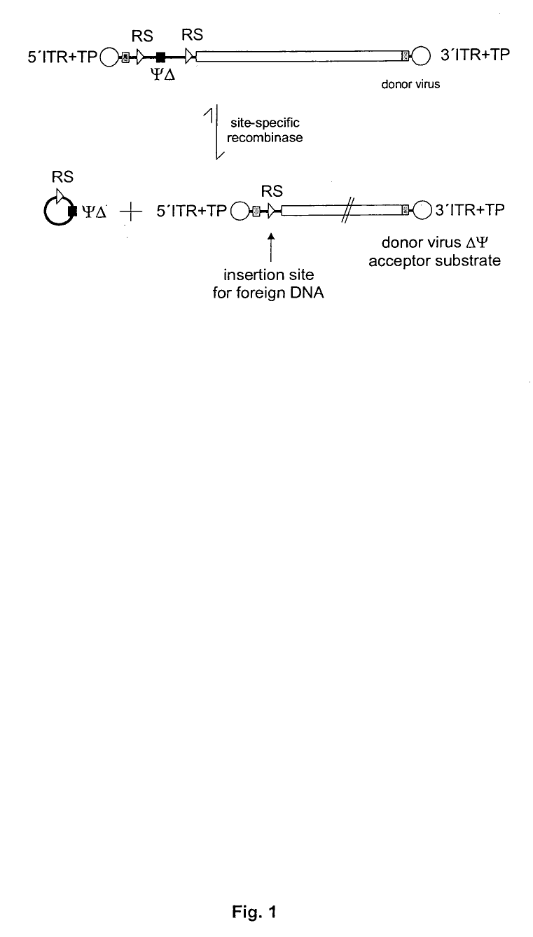

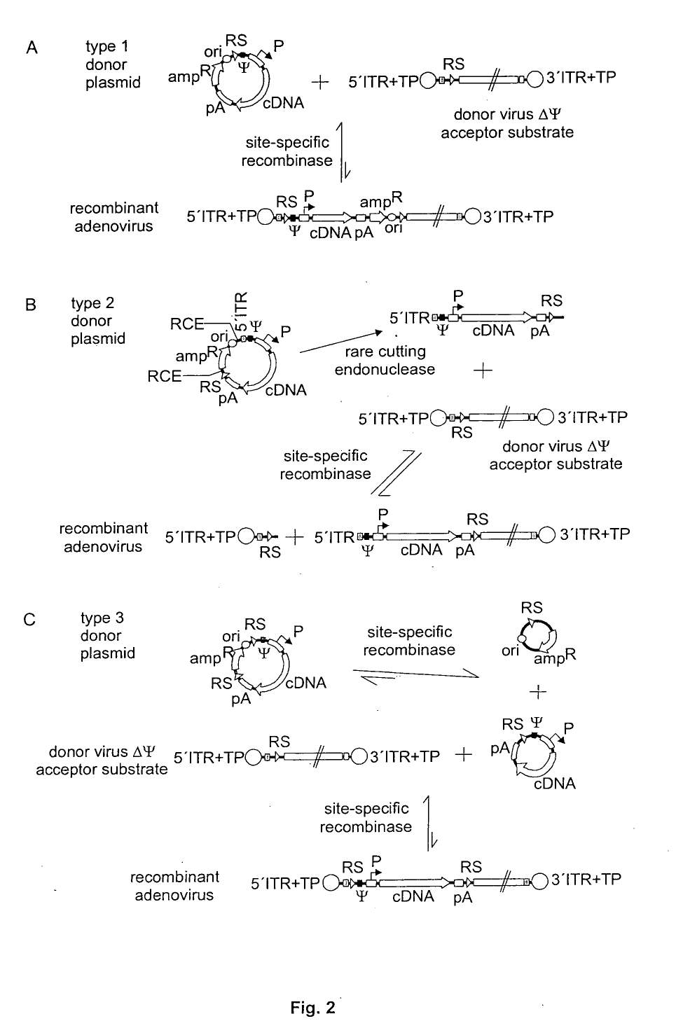

In accordance with the invention-related novel system for rAd generation, the packaging cell line is initially infected with the donor virus. Through the corresponding recognition sites in the donor virus genome, the partially deleted packaging signal of the donor virus is excised by the site-specific recombinase, expressed from the packaging cell line. The donor virus (ΔΨ) acceptor substrate arises from that, which (i) cannot be packed any longer into viral capsids and (ii) due to the unique recognition site for the recombinase contain an insertion point for the site-specific insertion of foreign DNA (see FIG. 1). In order to achieve a sufficient processing of the donor virus in this case, a high expression level of the site-specific recombinase is required. Through transfection, the donor plasmid, with the transgene cassette to be inserted, or a complex donor plasmid population, with a great number of sequences in the context of the donor plasmid, is introduced into the cells. Different types of donor plasmids, which are only slightly different in their structure, then lead, through likewise slightly different reactions, to the formation of the rAd through site-specific insertion (excision/insertion or terminal exchange, see below and FIG. 2). Basically, by means of recognition site(s) for the site-specific recombinase, the donor plasmid or parts of that, with the transgene cassette or the gene library and the complete viral packaging signal, are inserted site-specifically into the insertion site of the donor virus ΔΨ acceptor substrate. The rAd thus formed contain the transgene cassette or the gene library and the complete viral packaging signal. Furthermore, they contain (as the donor virus ΔΨ acceptor substrate) the covalent linked TP at one or both ITR's, thus each individual insertion event leads to the rescue of an infectious and normally replicating rAd. A complex mixture of donor plasmids thus leads to the rescue of a likewise complex mixture of rAd. Finally, it is a significant property of the invention-related system for rAd generation that the rAd, in contrast to contaminating non-processed donor viruses, contain the complete viral packaging signal and are thus preferably packed into viral capsids.

According to structure of the donor plasmid, in the case of the invention-related method for rAd generation, different types of the site-specific insertion can be distinguished.

In the following, 3 preferred types of donor plasmids are described:

Donor plasmids of the type 1 contain
a bacterial backbone with a bacterial resistance gene and a bacterial replication origin,
the complete viral packaging signal, followed by
a polylinker for the insertion of foreign DNA and/or already inserted foreign DNA, or (framed by a promoter and a polyadenylation signal) a polylinker for the insertion of coding sequences and/or already inserted coding sequences and
a recognition site located before the viral packaging signal for the site-specific recombinase.

After the transfection into the packaging cell line infected with donor virus through the site-specific recombinase, the complete donor plasmid is inserted, via an insertion/excision equilibrium reaction, into the insertion point of the donor virus ΔΨ acceptor substrate. The resulting rAd contain two recognition sites for the site-specific recombinase (see FIG. 2A).

Donor plasmids of the type 2 contain
a bacterial backbone with a bacterial resistance gene and a bacterial replication origin,
the viral ITR and the complete viral packaging signal, followed by
a polylinker for the insertion of foreign DNA and/or already inserted foreign DNA, or (framed by a promoter and a polyadenylation signal) a polylinker for the insertion of coding sequences and/or already inserted coding sequences and
two recognition sites for a rare cutting restriction endonuclease with a recognition sequence more than 8 bp long, which frame the bacterial backbone, where one of the recognition sites lies directly adjacent to the viral ITR.

Before the transfection into the packaging cell line infected with donor virus, the clonal or complex population donor plasmid is digested with the rare cutting restriction endonuclease. Fragments are set free by this, which contain, in sequential sequence, the viral ITR, the complete viral packaging signal, the inserted foreign DNA and a single recognition sequence for the site-specific recombinase. The longer the recognition sequence of the rare cutting restriction endonuclease, the smaller is the probability of the occurrence of a corresponding sequence in the transgene cassette or individual sequences of the gene library, which would disturb the release of these fragments. After the transfection, the fragments are inserted through the site-specific recombinase via a terminal exchange reaction into the insertion site of the donor virus ΔΨ acceptor substrate. The resulting rAd contain only one recognition site for the site-specific recombinase (see FIG. 2B).

Donor plasmids of the type 3 contain
all elements of the donor plasmids of the type 1 and
a second recognition site for the site-specific recombinase, which is localized so that (i) both recognition sites are oriented in parallel and (ii) both recognition sites frame the bacterial backbone with replication-strain and bacterial-resistance genes.

After the transfection into the packaging cell line infected with donor virus, the bacterial backbone is initially excised by the site-specific recombinase. A circular DNA molecule is generated as a product, which contains the complete viral packaging signal, the foreign DNA to be inserted and an single recognition site for the site-specific recombinase. This is then inserted through the site-specific recombinase, via an insertion/excision equilibrium reaction, into the insertion site of the donor virus ΔΨ acceptor substrate. The resulting rAd contain two recognition sites for the site-specific recombinase (see FIG. 2C).

With employment of donor plasmids of the type 1 and 3, rAd is formed, where the inserted DNA and thus also the complete viral packaging signal is framed by two parallel repeated recognition sites for the site-specific recombinase. The rAd are thus a further substrate for the excision/insertion equilibrium reaction of the site-specific recombinase. By the excision, the entire inserted DNA is again excised, including the packaging signal. Thus the amplification of this rAd is done preferably on cells which do not express the site-specific recombinase. The selection against the contamination with unprocessed donor viruses is done here via the partially deleted packaging signal only.

In contrary, with employment of donor plasmids of the type 2, rAd are formed, which contain only one recognition site for the site-specific recombinase. They are not a substrate for the excision/insertion reaction but for the terminal exchange reaction. This is not associated with the loss of the packaging signal. rAd thus generated can be amplified both on the packaging cell line, which expresses the site-specific recombinase (selection against the contamination with unprocessed donor viruses (i) using the excision of the packaging signal through the site-specific recombinase and (ii) using the partially deleted packaging signal), as well as on cells which do not express these (selection against the contamination with unprocessed donor viruses only via the partially deleted packaging signal).

As a basis for the construction of the donor viruses, human or non-human adenoviruses are used, in order to generate correspondingly clonal or complex populations of recombinant human or non-human adenoviruses. Human adenoviruses are preferably used, for example the serotype 5 (Ad5). In order to achieve a high capacity of the donor viruses for the insertion of foreign DNA, donor viruses can be used, in which one/several non-essential gene(s) is/are deleted. Also one/several essential gene(s) can be deleted, which must then be made available in trans by the packaging cell line or the producer cells.

As producer cells for the amplification of the donor virus and/or the recombinant viruses derived from this, cells or cell lines are used, which are permissive for the corresponding, where appropriate, partially deleted recombinant virus, for example the E1-complementing 293 cells for the amplification of E1-deleted Ad5-derived donor viruses or the clonal or complex populations of recombinant adenoviruses derived from these. The packaging cell line is obtained on the basis of the producer cell line through stable transfection of the gene for the site-specific recombinase. The expression of the recombinase gene can be constitutive or regulated. The recombinase genes can be a fusion gene from the recombinase gene and the coding sequences for a nuclear localization signal, in order to increase the concentration of the recombinase in the cell nucleus. As site-specific recombinases, it is preferable to employ recombinases of the Int family, for example the Cre recombinase or the Flp recombinase.

For the construction of clonal rAd populations, coding sequences, as well as elements, which control their expression (promoters, polyadenylation signals, among others) are used as transgene(s) in the donor plasmids. For the expression of one or several genes in cells, the sequence to be expressed is preferably provided with a promoter, which is either constitutively active or regulated. As promoters, viral or cellular promoters, or also combinations from both of them, can be used. The genome sequence or the cDNA of a gene can be used for the objective of a gene therapy, whose product in the case of the desease to be handled is missing, occurs in non-physiological quantities, or is defective. A part of a genome sequence can also be used, which spans a mutation in the target gene and can recombine homologously. For the objective of a tumor gene therapy, different genes can be used which cause a slowed-down growth or a killing of the tumor cells—where appropriate, in combination with remedies or through immunostimulation. For the objective of a vaccination, one or several possibly changed genes of the pathogenic organism can be used, against which a immunization should be achieved.

Invention-related, the formation of complex rAd populations is particularly favored. In case of the construction of complex rAd populations, with the objective of the construction of gene libraries, mixed populations of coding sequences are used in the donor plasmids, for example cDNA libraries from human or animal tissues or cells. This can be done, for example, with the objective of the isolation of new genes. With the construction of complex rAd populations, with the objective of the functional change of a known gene, mixed populations of mutated sequences of this gene are used in the donor plasmids. This can be used, for example, for the generation of gene-library variants of a protein (e.g. enzymes or antibodies), with the objective of a functional optimization of this protein. The coding sequences will be surrounded by elements which control their expression (promoters, polyadenylation signals). A further possible area of application of complex populations of rAd is the construction of libraries with non-coding or non-expressed sequences, for example, for the characterization or optimization of binding sites of DNA-binding proteins or enzymes.

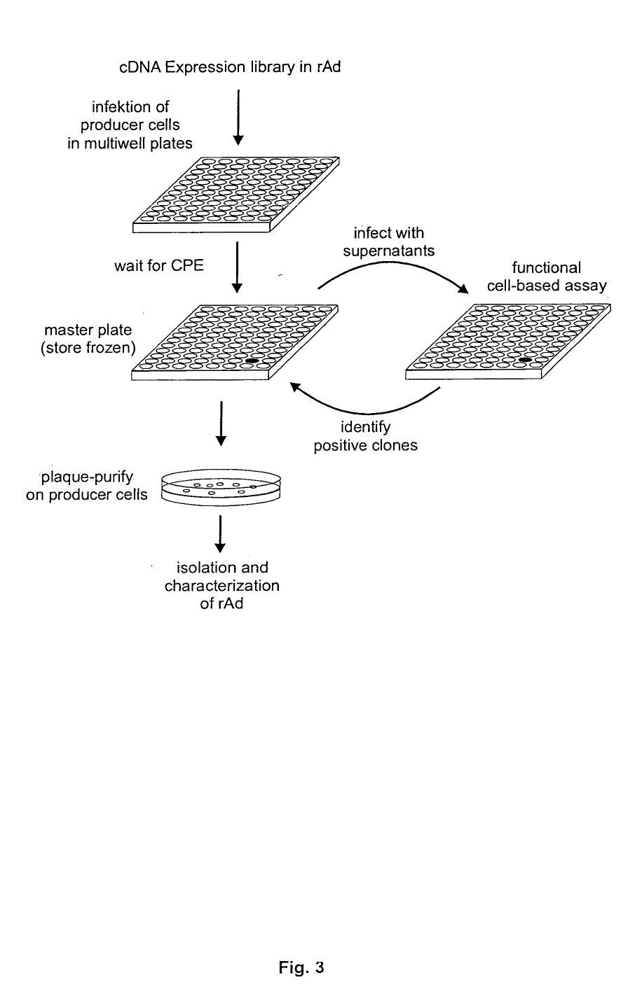

Provided that a cell-based test system is existing for the biological function searched for, the isolation of new genes with the properties searched for and/or the isolation of variants of a known gene with changed properties, can be done as follows: First of all, the titer of infectious particles in the the complex rAd population is determined. Then, for the generation of the so-called masterplates, producer cells in multiwell plates are infected, with a defined, low number of infectious particles per well. After the infection of the producer cells is completed, a freeze/thaw lysate of the masterplates is generated. Due to the stability of rAd, the masterplates can be frozen and stored. The set free amplified viruses are located in the supernatant of the wells. These supernatants can be used for the infection of the cells of the cell-based functional test system. The wells of the masterplates can then be identified, whose supernatants contain rAd, which cause the required phenotype after infection in the test system. Through plaque purification on producer cells, the rAd can be then be obtained from these supernatants in clonal form and finally the containd gene(s) can be characterized (see FIG. 3). With the invention-related system for the generation of recombinant adenoviruses, both clonal recombinant adenovirus populations, as well as complex recombinant adenovirus populations, can be generated. By a clonal population is meant a population, in which the same foreign DNA is integrated into all adenoviruses associated with the population. By foreign DNA is meant every DNA, which is not adenovirus DNA. A complex population, which is designated also as a complex mixed population, contains different adenoviruses which are distinguished in that they contain different foreign DNA in each case. Preferably, a complex recombinant adenovirus population contains at least two types of recombinant adenoviruses, which contain different foreign DNA in each case, in particular at least 10 different types, and the most preferred at least 100 different types.

In the invention-related donor virus, the packaging signal is partially deleted, so that a replication of the donor virus (without donor plasmid) in the packaging cell line is hampered, decreased or impaired. In this way, the desired rAd can be selectively amplified and thus selected for, with respect to the donor virus. In this case, the packaging signal in the donor virus is preferably at least 10%, in particular at least 20% and particularly preferred at least 30% and to up to 100% deleted, more preferably deleted up to 90% and particularly preferred deleted up to 70% (% means here the number of the deleted bases with reference to the total base number of the packaging signal).

The invention is explained further by the enclosed figures and the following implementation examples.

FIG. 1 shows the donor virus structure and formation of an donor virus ΔΨ acceptor substrate in a packaging cell line, which expresses the site-specific recombinase (gray box: Viral inverted terminal repeats (ITR's); black box: Partially deleted packaging signal (ΔΨ); white triangles: Recognition sites for the site-specific recombinase (RS); white circles: Viral terminal protein (TP)).

FIG. 2 shows the general structure of the preferred donor plasmids of the type I (A), type II (B), and type III (C), as well as the principle of the recombinant adenovirus generation through site-specific recombination with the donor virus ΔΨ acceptor substrate in a packaging cell line, which expresses the site-specific recombinase (gray box: viral inverted terminal repeats (ITR's); black box: complete viral packaging signal (T); white triangles: Recognition sites for the site-specific recombinase (RS); white circles: Viral terminal protein (TP); Arrow: Promoter (P); pA: Polyadenylation signal; RCE: Recognition site for a rare cutting endonuclease).

FIG. 3 shows a schematic overview of the utilization of adenovirus cDNA expression libraries for the identification of genes which induce a given phenotype in a functional cell-based assay.

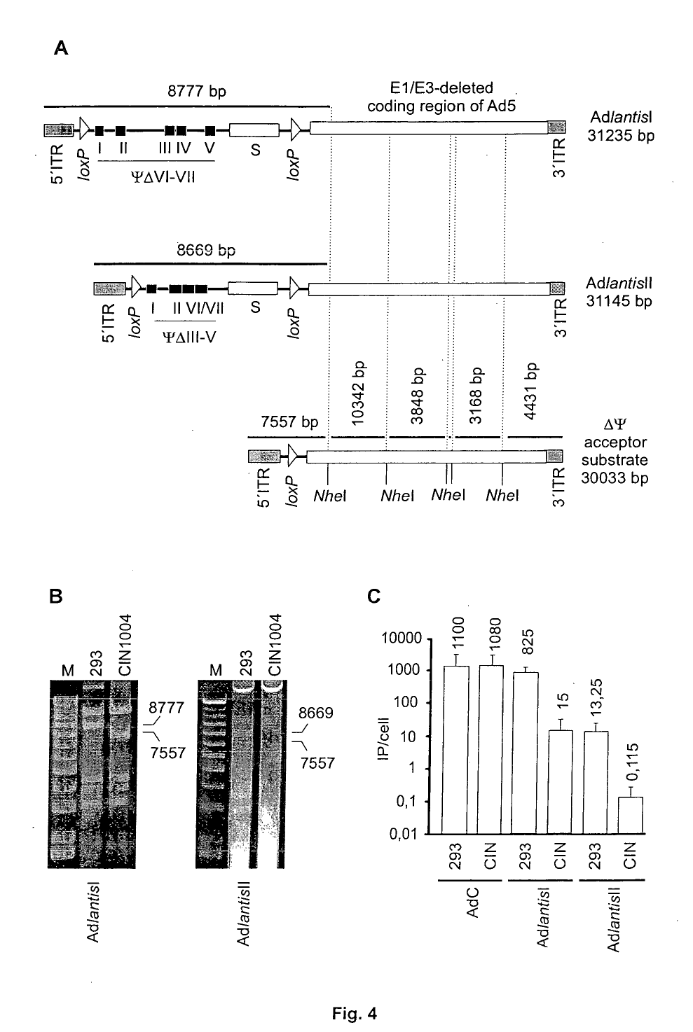

FIG. 4 shows the genome structures of the donor viruses AdlantisI and AdlantisII, which are a part of a system for the construction of clonal or complex populations of recombinant E1-deleted adenovirus serotype 5, and their functional characterization.

(4A) Schematic structure of AdlantisI and AdlantisII, as well as the donor virus ΔΨ acceptor substrate formed by excision of the packaging signal provided by CrelloxP, and recognition sites for Nhe I which were used in the analyses in (4b) (gray box: viral inverted terminal repeats (ITR's); black boxes with roman numbers: So-called A repeats of the partially deleted packaging signals (ΔΨ); white triangles: Recognition sites for Cre-recombinase (loxP); S: 929 bp spacer; gray box: Inverted terminal repeats of Ad5 (ITR's).

(4B) Verification of the highly efficient CrelloxP-mediated processing of the donor viruses to the donor virus ΔΨ acceptor substrate after infection of the packaging cell line CIN1004. As control, CIN 1004 cells and 293 cells were infected with AdlantisI or AdlantisII. Then the viral DNA was isolated and subjected to a restriction digestion with NheI. In case of both donor viruses, after infection of 293 cells, the 5′-terminal fragments characteristic for the unprocessed donor were observed, after infection with CIN1004 cells, on the other hand, exclusively the 5′-terminal fragment characteristic for the donor virus ΔΨ acceptor substrate (7557 bp) was obeserved. This indicates an almost complete processing of the donor viruses in the packaging cell line.

(4C) Verification of the growth reduction of the donor viruses on the packaging cell line CIN1004 as a result of the processing to the donor virus ΔΨ acceptor substrate. In each case 10⁶ CIN1004 cells or 293 cells as control were infected with AdlantisI or AdlantisII. After occurrence of the cytopatic effect, the number of the infectious particles formed per cell as progeny (IP) was determined by means of titration. In case of both donor viruses the number of IP formed per cell was lower on CIN1004 cells by approx. two orders of magnitude than on 293 cells. Furthermore, in case of AdlantisII, the number IP's formed per cell on both cell lines in total was approx. two orders of magnitude lower. AdC, a recombinant adenovirus, served as a further control, whose packaging signal is not flanked by loxP recognition sites and thus does not show any growth reduction on CIN1004 cells.

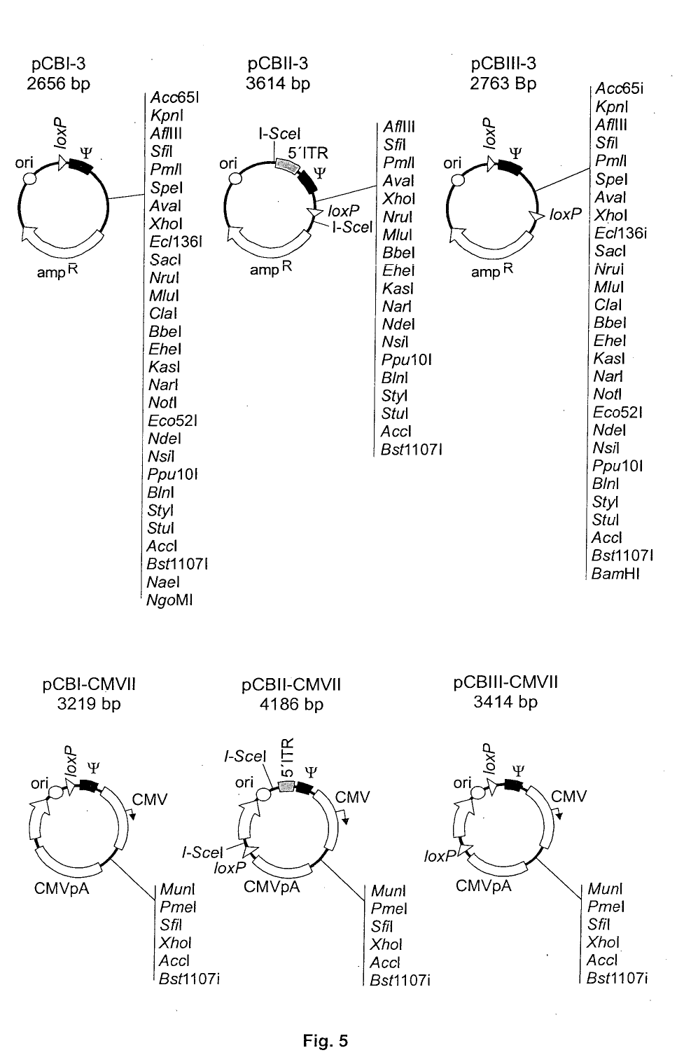

FIG. 5 shows the structures of the donor plasmids pCBI-3, pCBII-3, pCBIII-3, pCBI-CMVII, pCBII-CMVII and pCBIII-CMVII, which are part of a system for the construction of clonal or complex populations of recombinant E1-deleted adenovirus serotype 5, as well as their polylinkers for the insertion of DNA (white circles: Bacterial replication origin (ori); Amp^{R}: Ampicillin resistance gene; gray box: 5′ inverted terminal repeat of Ad5 (5′ITR); black box: Complete packaging signal of Ad5-content so-called A repeats I-VII (Ψ); white triangles: Recognition sites for the Cre-recombinase (loxP); I-SceI: Recognition sites for I-SceI; CMV: hCMV immediate early promoter; CMVpA: hCMV polyadenylation signal).

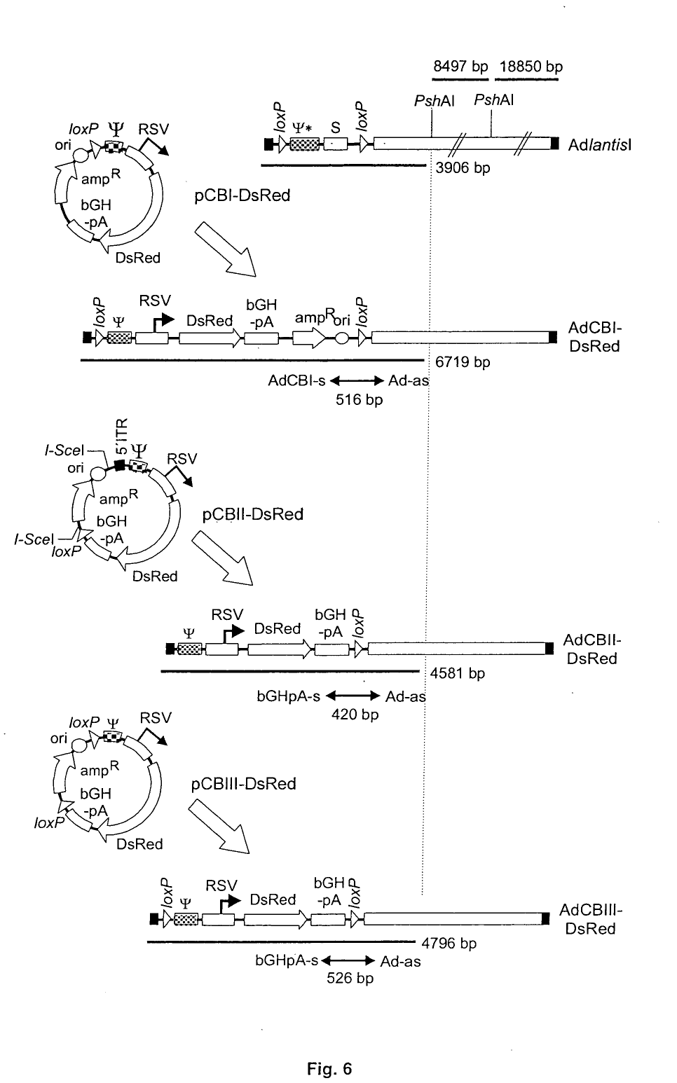

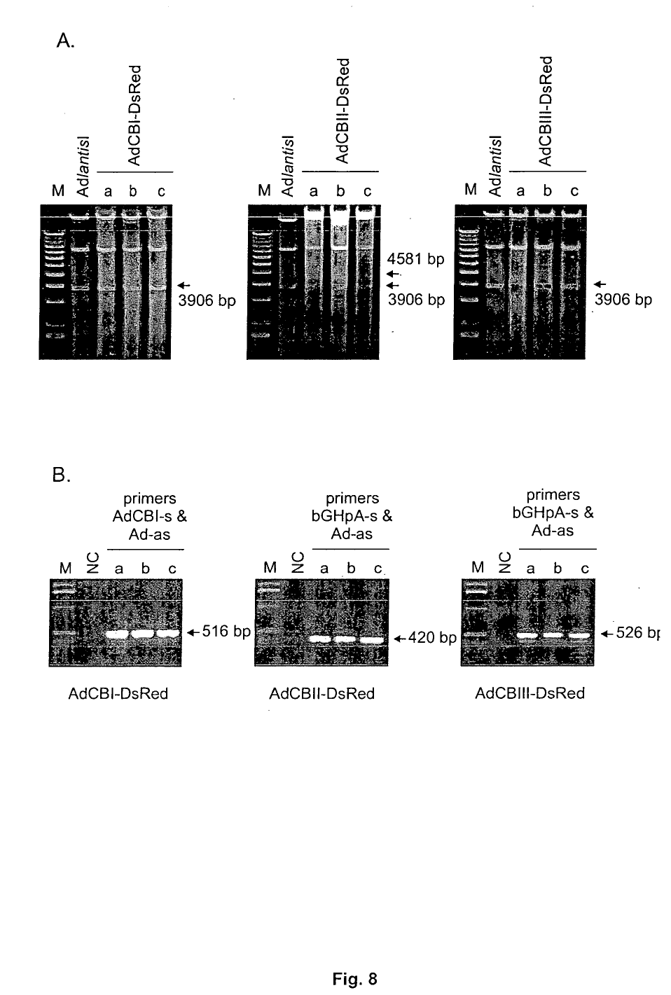

FIG. 6 shows the structure of the donor plasmids pCBI-DsRed, pCBII-DsRed and pCBIII-DsRed and the recombinant adenoviruses AdCBI-DsRed, AdCBII-DsRed and AdCBIII-DsRed formed from these donor plasmids by recombination with AdlantisI. Furthermore, the size of the PshAI fragments, in particular those of the 5′-terminal PshAI fragments, which served during the analysis in FIG. 8A for the distinction between viral DNA from AdlantisI and the newly formed recombinant adenoviruses. Furthermore, the binding sites of the primers and the sizes of the corresponding PCR products are indicated with the structures of the recombinant adenoviruses, whose formation in FIG. 8B proved the rescue of the recombinant adenoviruses (white circle (ori): bacterial replication origin; white triangle (loxP): loxP recognition site, Ψ: Complete packaging signal of Ad5; Ψ*: Partially deleted packaging signal of Ad5; black boxes: Inverted terminal repeats of Ad5 (ITR's); 5: Spacer; RSV: RSV-Promoter; bGHpA: Bovine growth hormone polyadenylation signal; DsRed: Open reading frame of the DsRed reporter gene).

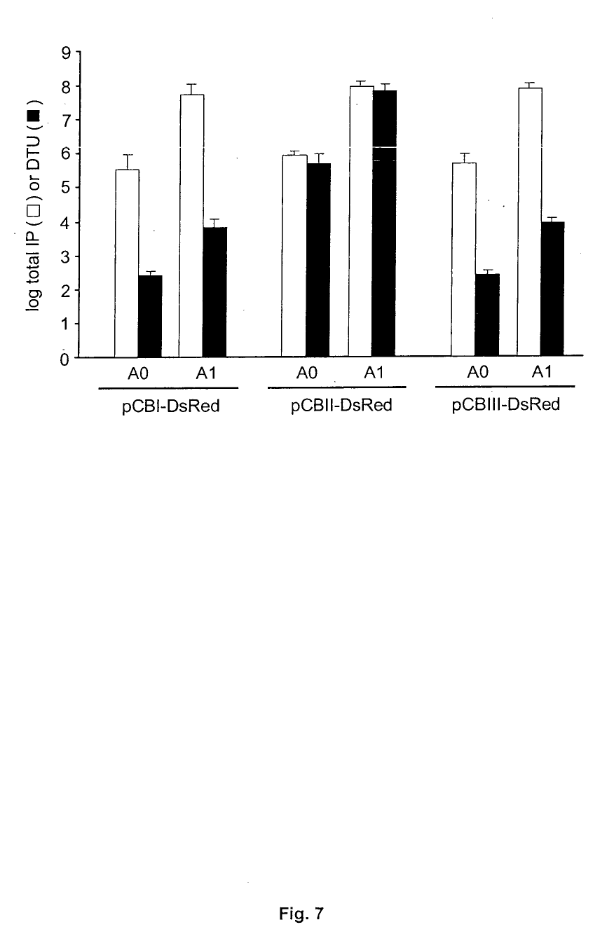

FIG. 7 shows the analysis of the mixtures obtained from residual donor virus and newly formed recombinant adenoviruses, with employment of the donor virus AdlantisI and the donor plasmids pCBI-DsRed, pCBII-DsRed or pCBIII-DsRed. In each case 10⁶ CIN1004 cells were infected with 5 infectious particles AdlantisI per cell and then transfected in each case with 10 μg pCBI-DsRed, pCBII-DsRed (1-SceI digested) or pCBIII-DsRed. For every donor plasmid, three completely independent experiments each were carried out. After occurrence of the virus-induced cytopathic effect (CPE) freeze/thaw lysates of the cells were generated (amplification round 0, A0). In each case 10⁶ CIN1004 cells were then infected with 1 ml A0 each. After occurrence of the CPE, freeze/thaw lysates were again generated (amplification round 1, A1). Then the total number of infectious particles in A0 and A1 (IP, white columns) was determined by dilution end-point analysis on 293 cells. Furthermore, the total number of newly-formed recombinant adenoviruses was determined in A0 and A1 as a total number of DsRed-transducing units (black bars, DTU). The mean value in each case from the three independent experiments, as well as the standard deviation, are indicated.

FIG. 8 shows the analysis of the mixtures of residual donor virus and newly formed recombinant adenoviruses on the level of the viral DNA, obtained with employment of the donor virus AdlantisI and the donor plasmids pCBI-DsRed, pCBII-DsRed or pCBII-DsRed. In each case, 1 ml of freeze/thaw lysate of the amplification round 1 (A1, for whose generation see FIG. 7) was used for the infection of 10⁶ 293 cells. After 36 hours, the replicated viral DNA was isolated from the infected cells through Hirt extraction. In each case, 3 independent experiments were performed per donor plasmid (a, b, c).

(8A) shows the digestion of 1 μg each of the Hirt extracts with PshAI. As control, viral DNA from donor virus AdlantisI was used. This digestion enables the distinction of the 5′-terminal fragments of the newly formed recombinant adenoviruses and the donor virus AdlantisI (see FIG. 6). With employment of pCBI-DsRed and pCBIII-DsRed as a donor plasmid, only the 3909 bp large 5′-terminal fragment of AdlantisI can be identified. With employment of pCBII-DsRed as a donor plasmid, both the 4581 bp sized 5′-terminal fragment of AdCBII-DsRed, as well as the 3909 bp sized 5′-terminal fragment of AdlantisI in a ratio of approx. 1:1 can be identified. This indicates that the formation of AdCBII-DsRed from pCBII-DsRed is far more efficient than that of AdCBI-DsRed from pCBI-DsRed and/or that of AdCBIII-DsRed from pCBIII-DsRed.

(8B) shows the PCR verification of the DNA of the newly formed recombinant adenoviruses of AdCBI-DsRed, AdCBII-DsRed or AdCBIII-DsRed in the Hirt extracts. In each case, 1 μl of the Hirt extract was used in a PCR with the indicated primers AdCBI-s or bGHpA-s and Ad-as. 1 μl H₂O served as negative control. Concerning the binding sites of the primers and the size of the corresponding PCR products, see FIG. 6 (M: DNA size marker). The occurrence of PCR products characteristic for the newly formed recombinant adenoviruses, with employment of all Hirt extracts, indicates that (even if the efficiency is different (cf. FIG. 8A)) recombinant adenoviruses arise from all three donor plasmids.

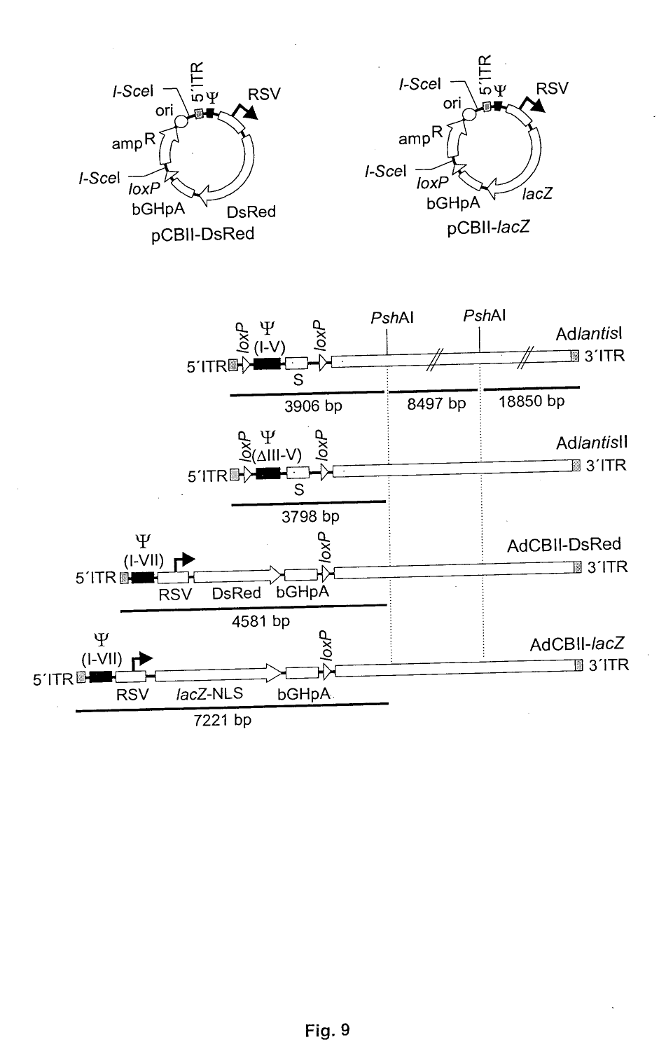

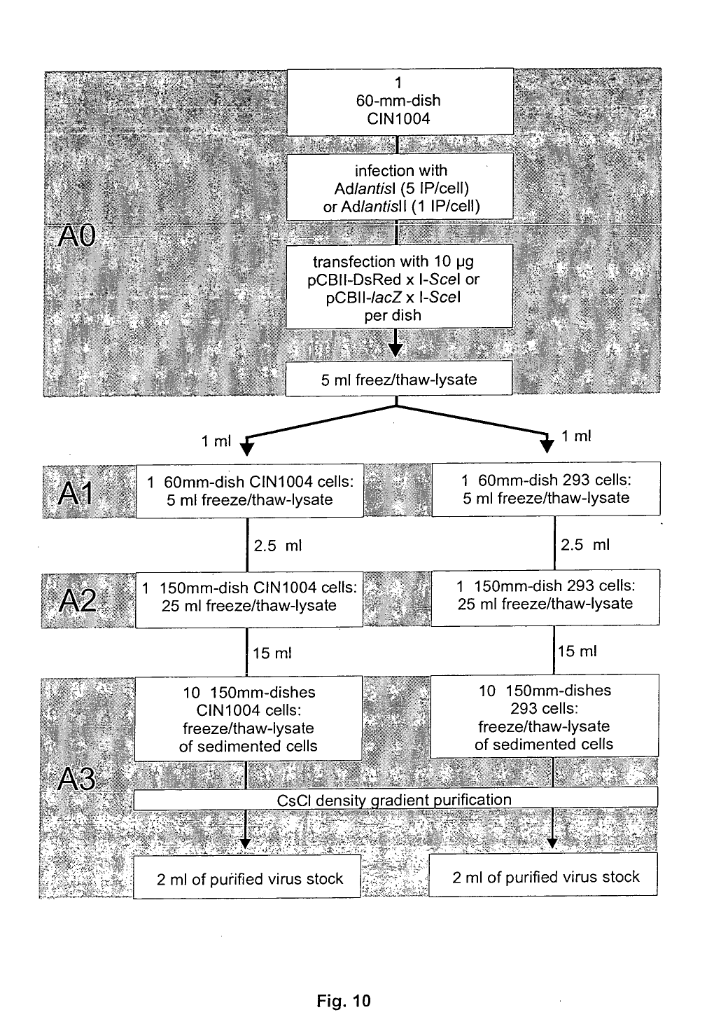

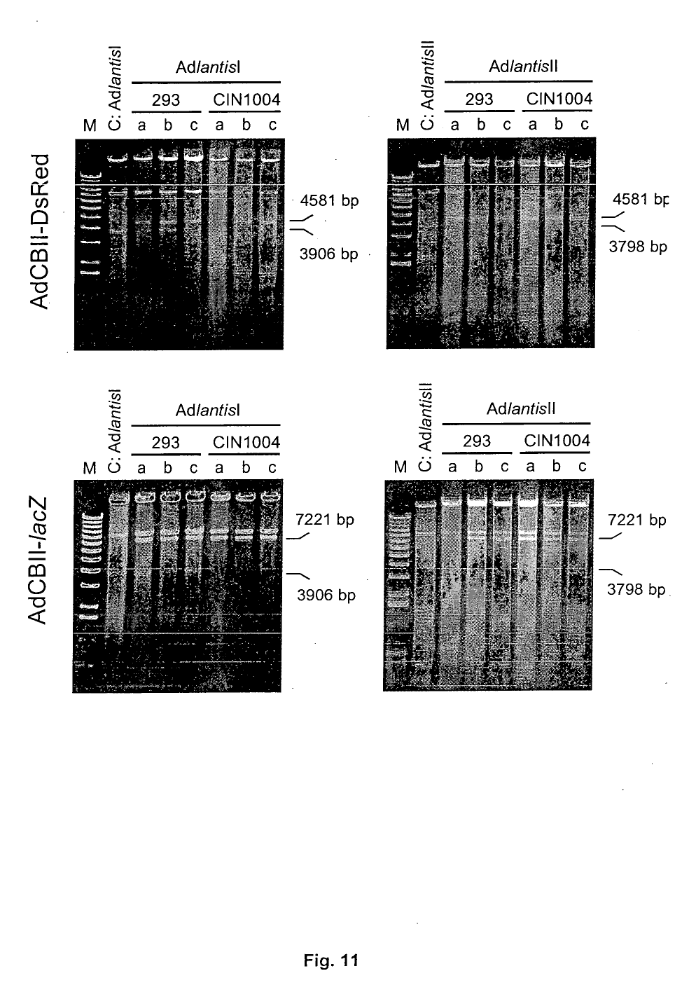

FIG. 9 shows the structure of the donor plasmids pCBII-DsRed and pCBII-lacZ and the recombinant adenoviruses AdCBII-DsRed and AdCBII-lacZ formed from these donor plasmids by recombination with the donor viruses of AdlantisI and AdlantisII. Furthermore, the size of the PshAI fragments is indicated, in particular that of the 5′-terminal PshAI fragments, which were used for the distinction between viral DNA of the donor viruses and the newly formed recombinant adenoviruses within the restriction analyses in the FIGS. 10 and 11.

FIG. 10 shows the experimental schematic that was used in the generation of large scale preparations of the recombinant adenoviruses AdCBII-DsRed and AdCBII-lacZ. According to this schematic, 3 parallel independent experiments were carried out in each case for both donor viruses AdlantisI and AdlantisII, in combination with the donor plasmids pCBII-DsRed or pCBII-lacZ.

FIG. 11 shows the analysis of the virus mixtures which were received in the amplification round 1 (A1), according to the schematic of FIG. 10. 1 ml of the freeze/thaw lysates A1 each was used for the infection of 10⁶ 293 cells. After occurrence of the cytopatic effect, the replicated viral DNA was isolated through Hirt extraction. In each case, 1 μg of the Hirt extract was then digested with PshAI. This enzyme generates characteristic fragments of the 5′-end of the donor viruses AdlantisI and AdlantisII, as well as of the recombinant adenoviruses AdCBII-DsRed and AdCBII-lacZ (see FIG. 9). As control (C), 1 μg each of purified DNA of the AdlantisI or AdlantisII donor virus was used. The upper two illustrations show the results with pCBII-DsRed as a donor plasmid (recombinant adenovirus AdCBII-DsRed), the two lower show those with pCBII-lacZ (recombinant adenovirus AdCBII-lacZ). In the right-hand illustrations AdlantisI was used as a donor virus, in the left-hand illustration AdlantisII. Within the illustrations, the three independent experiments each (a, b, c) are compiled with amplification on 293 cells or CIN1004 cells. In all approaches, the existence of the newly formed recombinant adenoviruses is identified by means of the characteristic 5′-terminal fragments (4581 bp with AdCBII-DsRed and 7221 bp with AdCBII-lacZ, see FIG. 9). With employment of AdlantisI and amplification on 293 cells, the 5′-terminal fragment of the donor virus can be additionally identified, which indicates a residual contamination with this donor virus. On the other hand, with employment of AdlantisII and amplification on 293 cells, the 5′-terminal fragment does not occur. In case of amplification on CIN1004 cells, with the employment of both donor viruses, the 5′-terminal fragment of the donor virus could also not be detected.

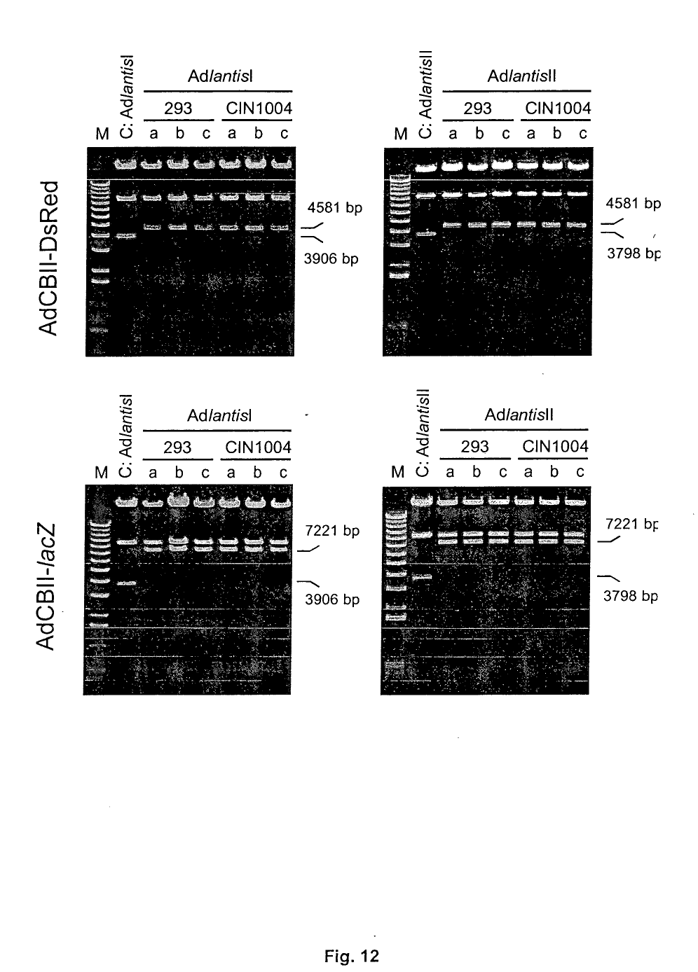

FIG. 12 shows the analysis of large scale preparations of the recombinant adenoviruses AdCBII-DsRed and AdCBII-lacZ, which were obtained according to the experimental schematic of FIG. 10. The viral DNA was extracted from the purified infectious particles and, in each case, 1 μg of the purified DNA digested with PshAI. This enzyme generates characteristic fragments of the 5′-end of the donor viruses AdlantisI and AdlantisII, as well as of the recombinant adenoviruses AdCBII-DsRed and AdCBII-lacZ (see FIG. 9); As control (C) 1 μg each of purified DNA of the donor virus AdlantisI or AdlantisII was used. The upper two illustrations show the results with the recombinant adenovirus AdCBII-DsRed, the two lower those with the recombinant adenovirus AdCBII-lacZ. In the right-hand illustrations AdlantisI was used as a donor virus, in the left-hand illustrations AdlantisII. Within the illustrations, the three independent experiments (a, b, c) are compiled in each case with amplification on 293 cells or CIN1004 cells. In all purified virus DNAs, only the characteristic 5′-terminal fragment of the recombinant adenovirus can be identified, i.e. the 4581 bp fragment with AdCBII-DsRed and the 7221 bp fragment with AdCBII-lacZ.

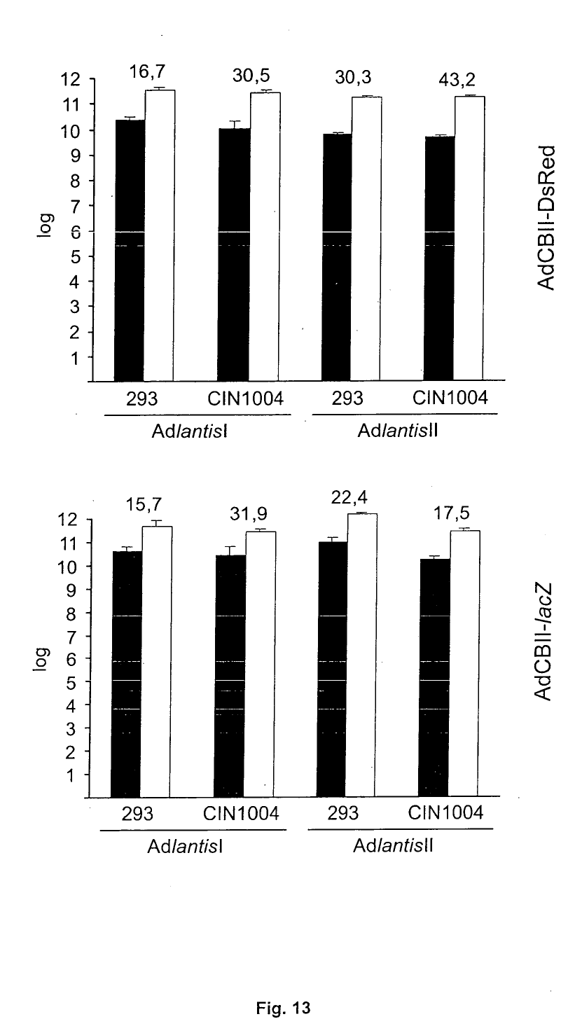

FIG. 13 shows the determination of the titers of intact infectious particles and the total titers of viral particles in the large scale preparations of AdCBII-DsRed and AdCBII-lacZ, which were generated according to the schematic in FIG. 10. The titer of intact infectious particles was determined by dilution end-point analysis on 293 cells (black column), the total titer of viral particles through measurement of the photometric absorption of the virus preparation (white column). The mean value of the three independent experiments and the standard deviation is indicated in each case. Above the column pairs, the ratio of total titer of viral particles to the titer of infectious particles is indicated.

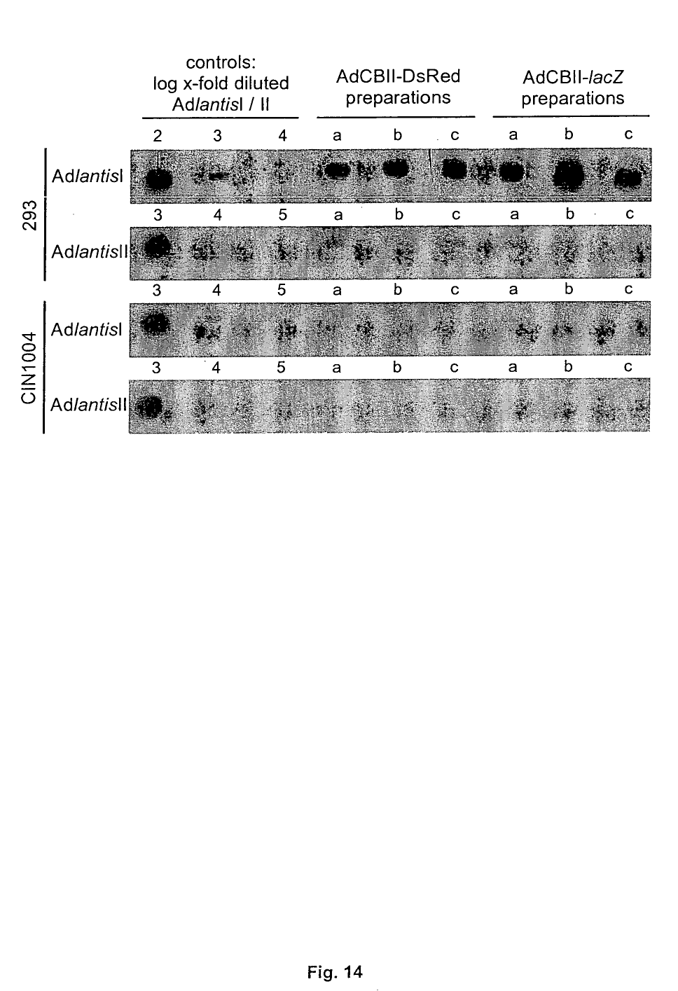

FIG. 14 shows the more precise determination of the extent of the residual contamination with donor viruses in the large scale preparations of AdCBII-DsRed and AdCBII-lacZ, which were generated according to the schematic of FIG. 10 through Southern Blot. In each case, 1 μg PshAI-digested viral DNA from the purified virus preparations was separated via agarose gels and transferred onto nylon membranes. The specific, radioactive detection of the 5′-terminal PshAI fragment of the donor viruses (AdlantisI: 3906 bp; AdlantisII: 3798 bp) was done with a labled probe, which identifies the spacer fragment (spacer, S in FIG. 9), which is containd in these donor viruses only, not, however, in the arisen recombinant adenoviruses AdCBII-DsRed and AdCBII-lacZ. To the left the cell line is indicated, on which the amplification of the viruses (A1-A3 in FIG. 10) was done, as well as the donor virus, which was used in the rescue of the viruses (A0 according to FIG. 10). As controls in each case 1 μg herring-sperm DNA with 0.01 μg (100× dilution, log 2), 0.001 μg (1000× dilution, log 3) 0.0001 μg (10,000× dilution, log 4) or 0.00001 μg (100,000× dilution, log 5) PshAI-digested DNA of the donor virus used in each case was used. The scale of the helper virus contamination can be estimated through comparison of the band intensity with the controls.

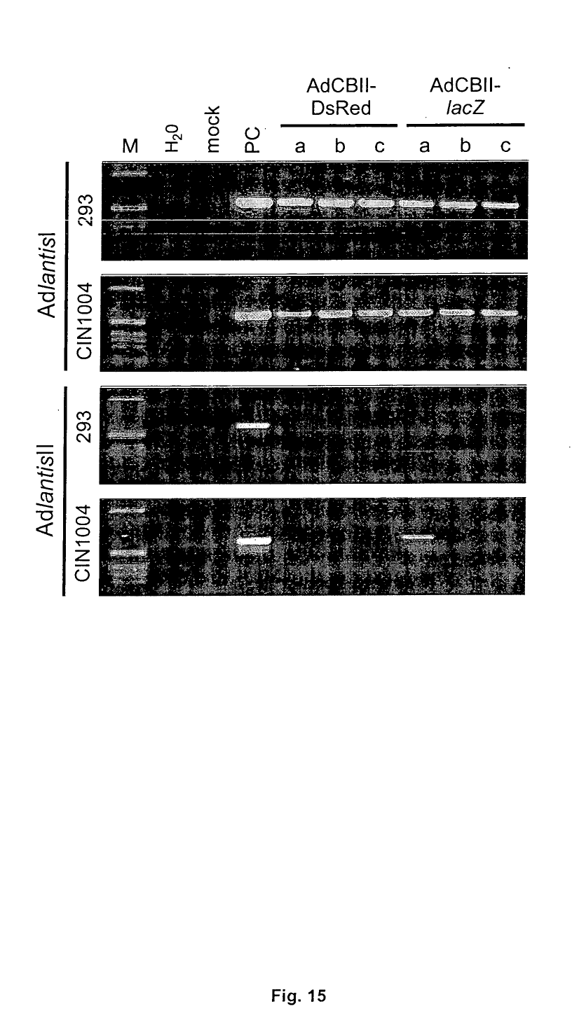

FIG. 15 shows the testing of the large scale preparations of AdCBII-DsRed and AdCBII-lacZ, which were generated according to the schematic of FIG. 10, on contamination with replication-competent wild type adenoviruses (RCA). In each case 10⁷ Huh7 cells were infected with 10⁸ infectious particles of the purified virus preparations. After 7 days, the cells were lysed through freeze/thaw lysis and ⅓ of the lysate used in each case for the further infection of 10⁷ Huh7 cells. After a further 7 days, the cell culture supernatant was tested by means of PCR for the existence of RCA. Primers were used in this case, which lead to the formation of a 600 bp product with the existence of RCA DNA. As negative controls (1) H₂O and (2) cell culture supernatant from non-infected Huh7 cells (mock) were used. Preparations of another recombinant adenovirus contaminated with RCA (M: DNA size marker) served as positive control (PC).

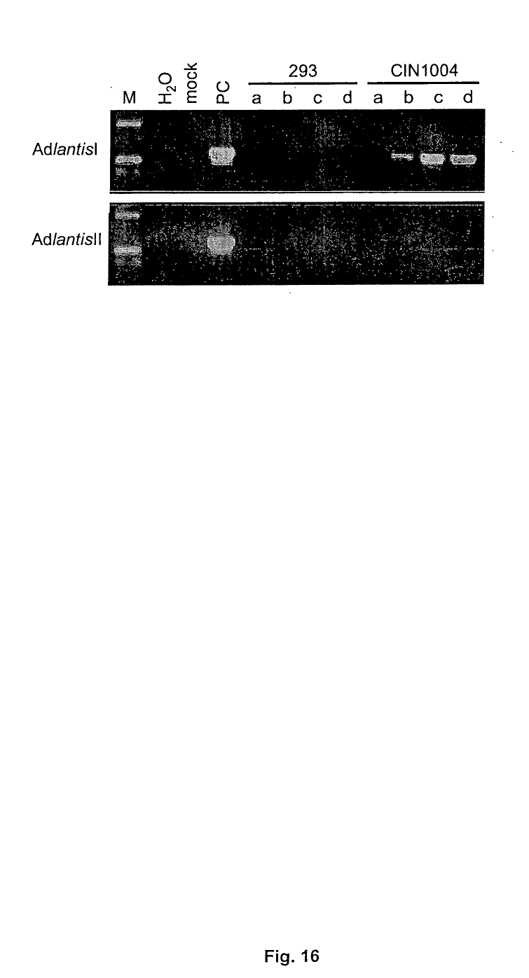

FIG. 16 shows the high efficiency with which replication-competent wild type adenovirus (RCA) is formed from AdlantisI, but not from AdlantisII, after infection of CIN1004 cells. In 4 independent experiments in each case (a, b, c, d) 293 cells or CIN1004 cells were infected with 5 infectious particles per cell of AdlantisI or 1 infectious particle per cell of AdlantisII. After occurrence of the virus-induced cytopathic effect, the cells were lysed through freeze-thaw lysis and the lysates tested by means of PCR for the existence of RCA. Primers were used in this case, which lead to the formation of a 600 bp product with the existence of RCA DNA. As negative controls H₂O or freeze/thew lysates of mock-infected 293 cells (mock) were used, as positive controls (PC) freeze/thaw lysates RCA-infected cells were used (M: DNA size marker).

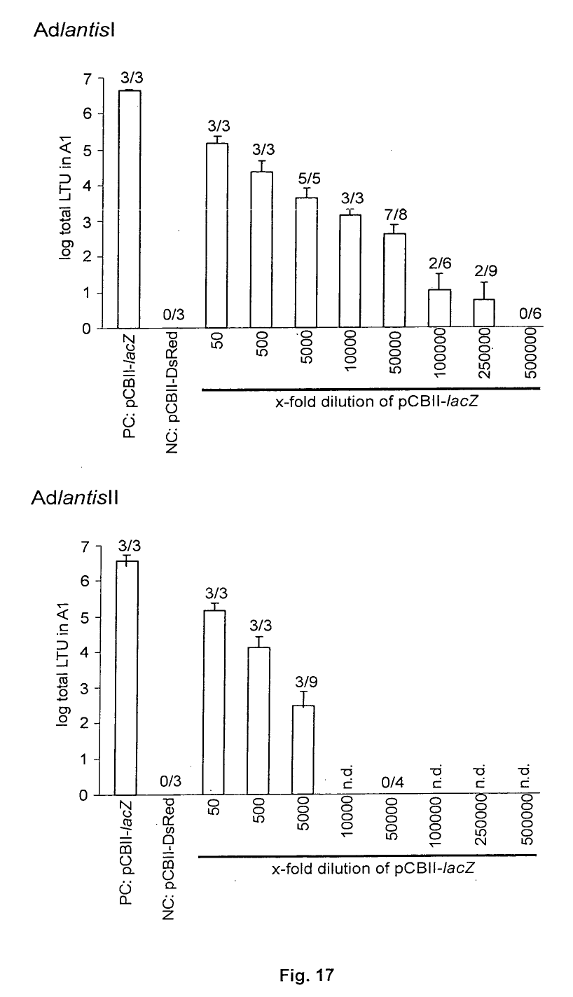

FIG. 17 shows the determination of the number of independent recombinant adenovirus clones which arise with employment of the donor viruses AdlantisI or AdlantisII and donor plasmids of the type 2 from 10⁶ CIN1004 cells. In each case 10⁶ CIN1004 cells were infected with 5 infectious particles per cell of AdlantisI (above) or 1 infectious particle per cell AdlantisII (below) and subsequently transfected with 12 μg, in each case, of different mixtures of 1-SceI-digested pCBII-DsRed and pCBII-lacZ. Mixture ratios of 50:1 to 500,000:1 were used in this case. Donor virus-infected CIN1004 cells transfected with 12 μg I-SceI-digested pCBII-lacZ served as positive controls, corresponding transfections with 12 μg I-SceI-digested pCBII-DsRed served as negative controls. After occurrence of the virus-induced cytopathic effect (CPE) freeze/thaw lysates of the cells were generated. ⅕ of these lysates were used in each case for the infection of 10⁶ 293 cells. After occurrence of the CPE freeze/thaw lysates of the cells were generated and in turn ⅕ of these lysates were used for the infection of 10⁶ Huh7 cells. After 48 hours, the Huh7 cells were stained with X-Gal. The number of lacZ transducing units (LTU) arising from pCBII-lacZ could then be determined through counting the blue-stained cells. The columns indicate the mean value of the total number on LTU in each case and the standard deviation in the experiments, where blue cells could be detected. Above the the columns, the ratio of the number of positive experiments versus the total number of experiments is indicated.

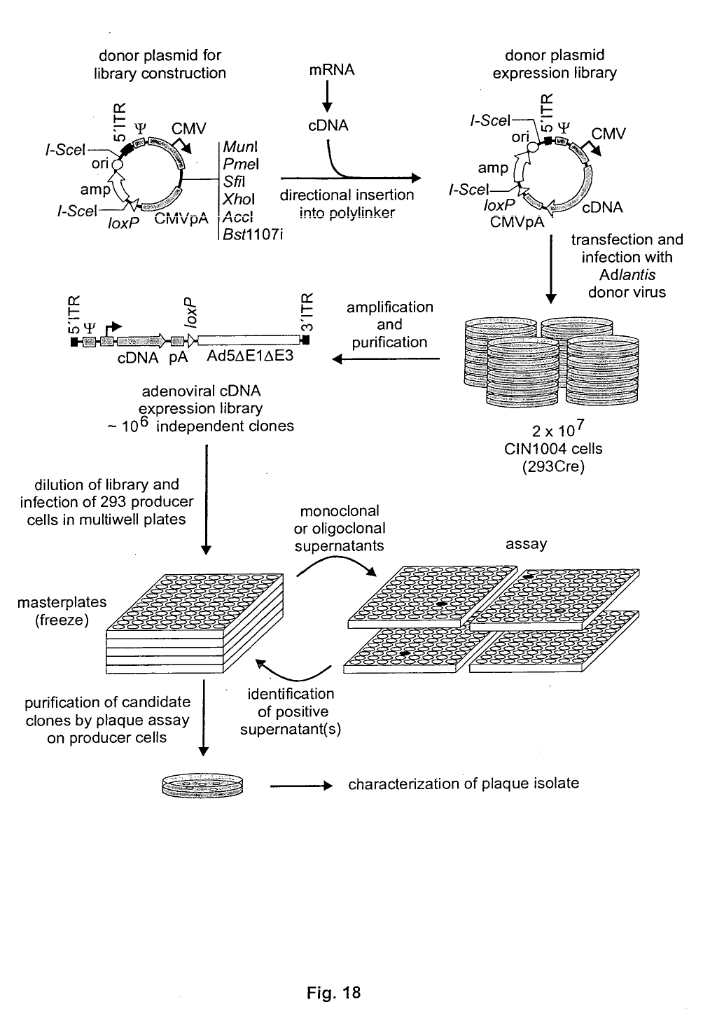

FIG. 18 shows the experimental schematic for the generation of adenoviral cDNA expression libraries, as well as their employment for the identification of genes, which cause a certain phenotype in a test system (white circle: Bacterial replication origin (ori); white arrow: Ampicillin resistance gene (amp); white triangle: Recognition site loxP (loxP); black boxes: inverted terminal repeats of Ad5 (ITR's); Ψ: Complete packaging signal of Ad5; Ad5ΔE1ΔE3: Coding sequences of Ad5 with deletion of the E1 and E3 region; Box with arrow: CMV promoter (CMV); pA: CMV polyadenylation signal).

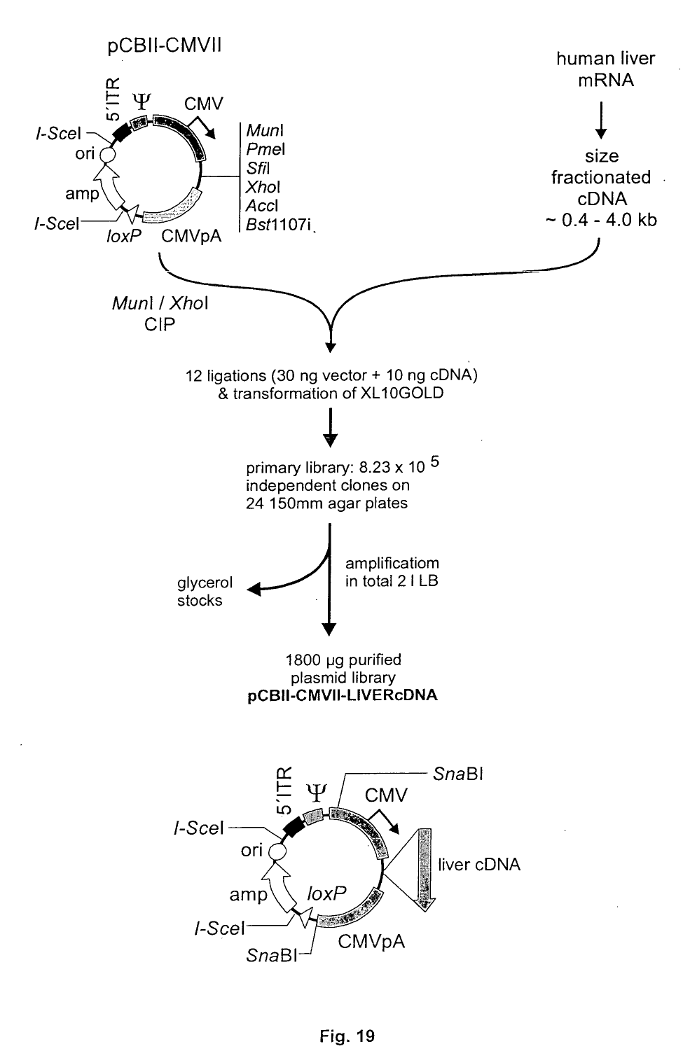

FIG. 19 summarizes the experimental procedure for the construction of the expression library for human liver cDNA in the donor plasmid pCBII-CMVII (white circle: Bacterial replication origin (ori); white arrow: Ampicillin resistance gene (amp); white triangle: Recognition site loxP (loxP) black box: 5′ inverted terminal repeat of Ad5 (5 ITR); Ψ: Complete packaging signal of Ad5; Box with arrow: CMV promoter (CMV); pA: CMV polyadenylation signal.

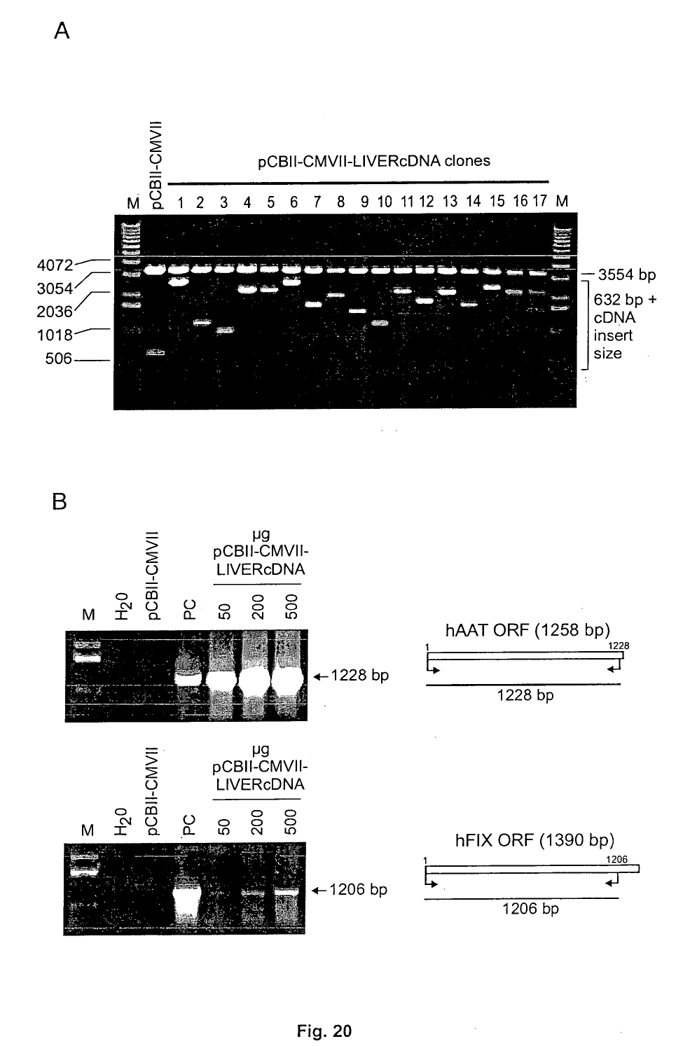

FIG. 20 shows the characterization of the expression library for human liver cDNA in the donor plasmid pCBII-CMVII (pCBII-CMVII-LIVERcDNA), which had been generated according to the schematic of FIG. 19.

(20A) shows the determination of the size range of the inserted cDNA's. In each case, 1 μg plasmid DNA from separated clones was digested with SnaBI. As control, pCBII-CMVII without inserted foreign DNA was digested with SnaBI. This enzyme delivers a 3554 bp fragment from the plasmid backbone, as well as a further fragment, which contains the expression cassette along with CMV promoter, inserted cDNA and CMV polyadenylation signal (see FIG. 19). From the size of this fragment, the size of the inserted cDNA can be estimated through subtraction of the sum of the sizes of the CMV promoter and the polyadenylation signal (632 bp).

(20B) shows the verification of the presence of the cDNA's for hAAT (above) and hFIX (below) by means of PCR. Besides the illustrations, the binding sites of the used primers, as well as the size of the products are schematically displayed. 50, 200 or 500 μg of the plasmid library were used in the PCR. H₂O and 10 ng pCBII-CMVII served as negative controls, 10 ng each of a plasmid with the complete reading frame of hAAT (above) or hFIX (below) served as positive controls (PC).

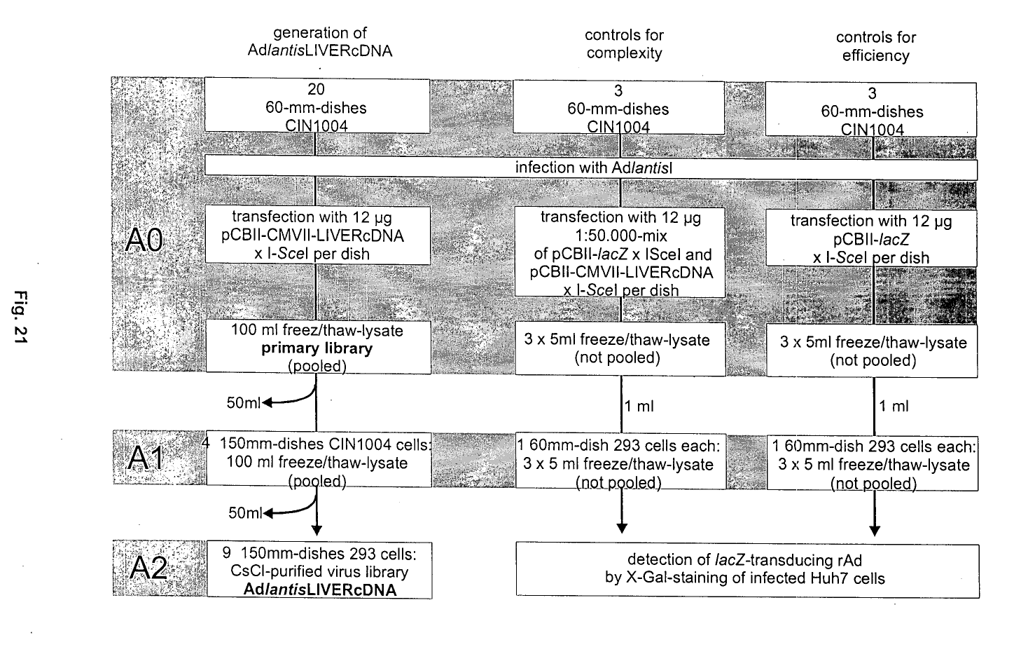

FIG. 21 shows the experimental schematic which was used in the conversion of the expression library for human liver cDNA into the donor plasmid pCBII-CMVII (“pCBII-CMVII-LIVERcDNA”) in adenoviral cDNA expression libraries.

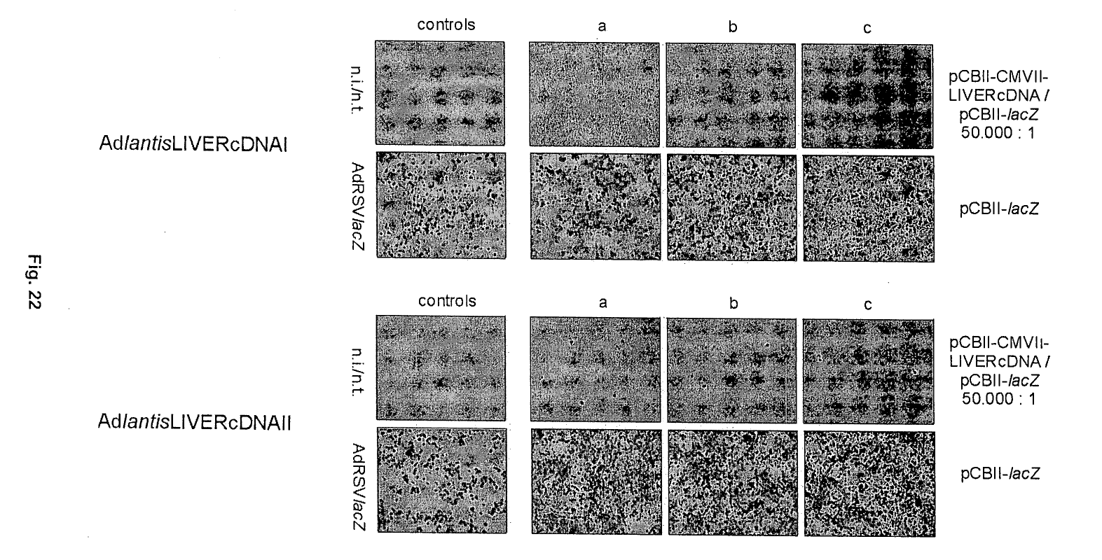

FIG. 22 shows the controls for complexity and efficiency of the virus rescue with the generation of the adenoviral liver cDNA expression libraries AdlantisLIVERcDNAI & II according to the schematic of FIG. 21. In each case, 1 ml of the freeze/thaw lysates of the A1 of the 3 (a, b, c) controls for complexity (pCBII-CMVII-LIVERcDNA/pCBII-lacZ 50.000:1) and efficiency (pCBII-lacZ) were used for the infection of subconfluent Huh7 cells in 60 mm cell culture dishes. After 48 hours the cells were stained with X-Gal.

Non-infected and non-transfected Huh7 cells (n.i./n.t.) served as negative controls, while Huh7 cells, which have been infected with 20 infectious particles per cell of a recombinant adenovirus with a RSV-promoter-driven expression cassette lacZ (Ad RSV-lacZ), served as positive controls.

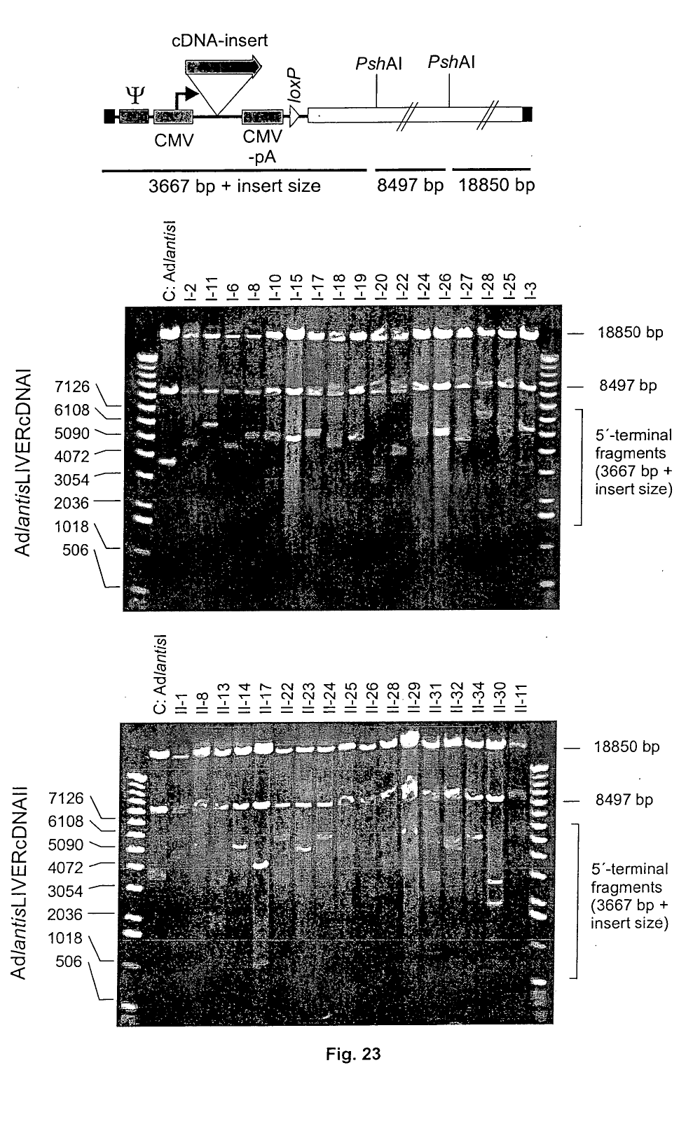

FIG. 23 shows the characterization of the adenoviral liver cDNA expression libraries AdlantisLIVERcDNAI & II concerning sizes of the inserted cDNAs. Individual virus clones, which were isolated by plaque assay on 293 cells, were used for the infection of 293 cells in each case. After 36 hours, the replicated viral DNA was extracted and subjected to a restriction analysis with PshAI. This enzyme supplies from the 5′-end of the recombinant adenoviruses a characteristic fragment whose size consists of 3667 bp vector sequences plus the size of the inserted cDNA.

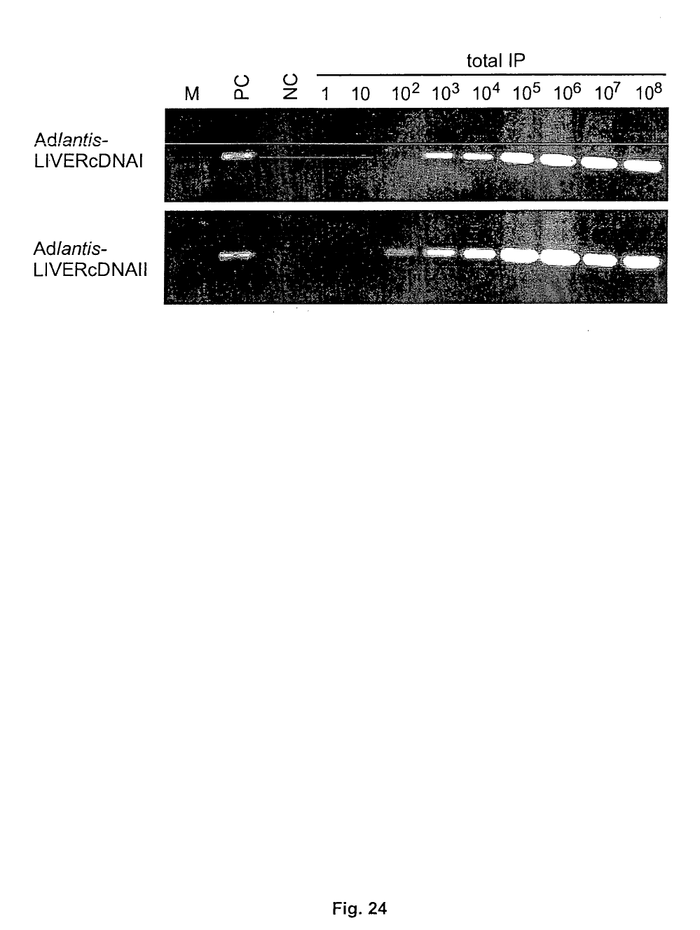

FIG. 24 shows the determination of the extent of contamination of the adenoviral liver cDNA expression libraries AdlantisLIVERcDNAI & II with replication-competent adenoviruses (RCA). In each case, 10⁷ Huh7 cells were infected with 1-10⁸ infectious particles (IP) of the expression libraries. After 7 days the cells were lysed through freeze/thaw lysis and, in each case, ⅓ of the freeze/thaw lysate was used for the further infection of 10⁷ Huh7 cells.

After a further 7 days the cell culture supernatant was tested by means of PCR for the existence of RCA. Primers were used, which lead to the formation of a 600 bp product with the existence of RCA DNA. The RCA contamination for AdlantisLIVERcDNAI is less than 1%, for AdlantisLIVERcDNAII less than 10%. A preparation of another recombinant adenovirus contaminated with RCA served as positive control (PC). Cell culture supernatant from non-infected Huh7 cells was used as negative control (NC) (M: DNA size marker).

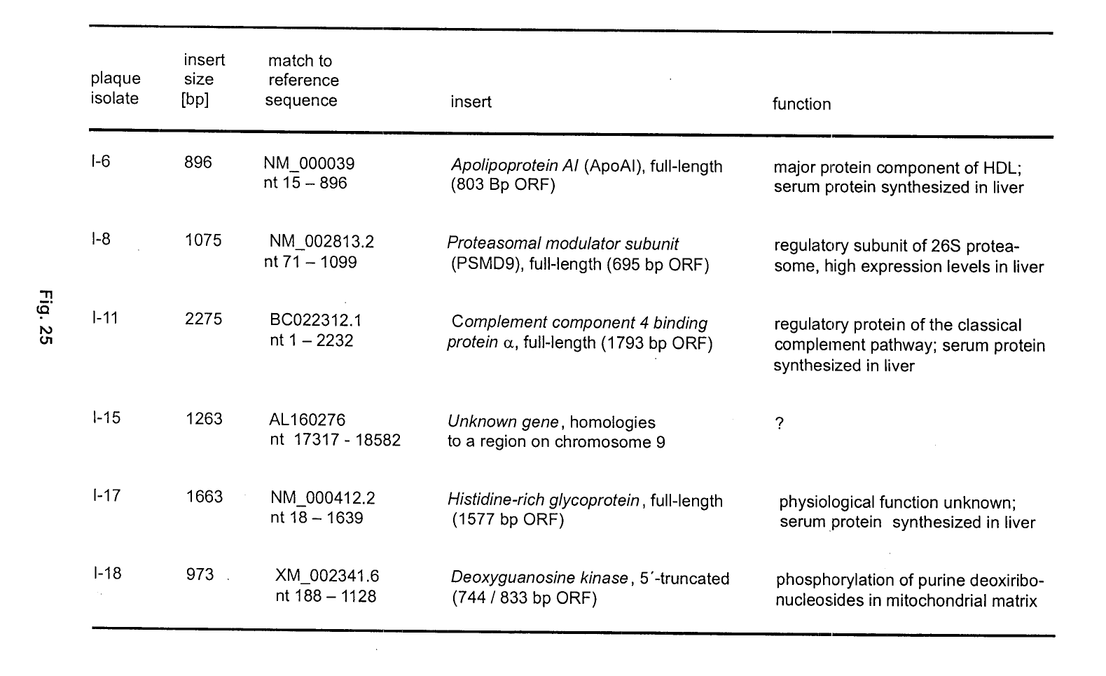

FIG. 25 shows in tabular form the characterization of the inserted cDNA's in clones 1-6, 1-8, 1-9, 1-15, 1-17 and 1-18, isolated by plaque assay from the adenoviral liver cDNA expression library AdlantisLIVERcDNA I.

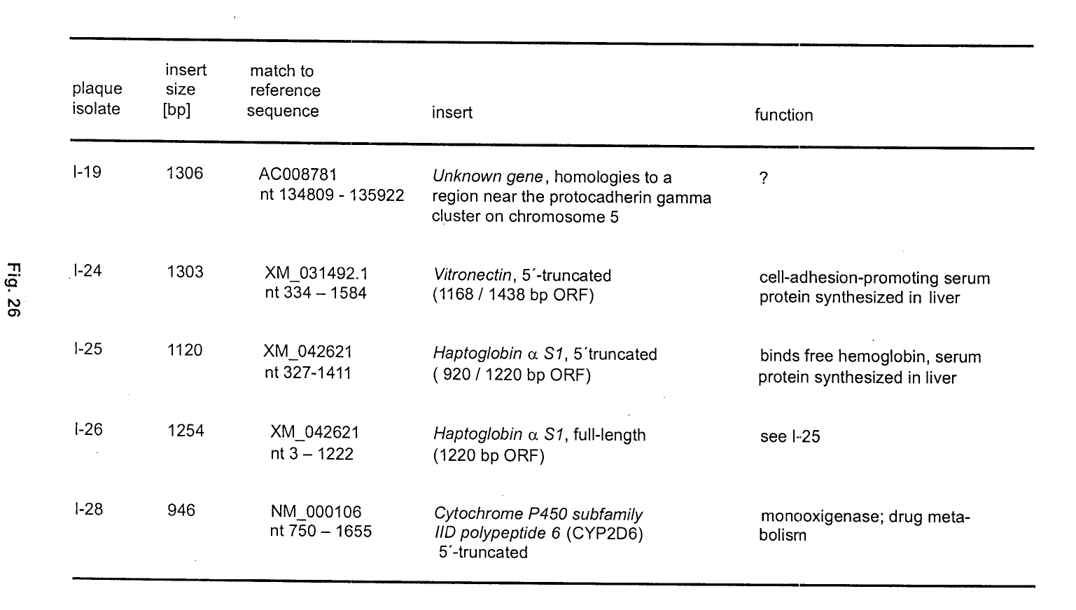

FIG. 26 shows in tabular form the characterization of the inserted cDNA's in clones 1-19, 1-24, 1-25, 1-26 and 1-27, isolated by plaque assay from the adenoviral liver cDNA expression library AdlantisLIVERcDNA I.

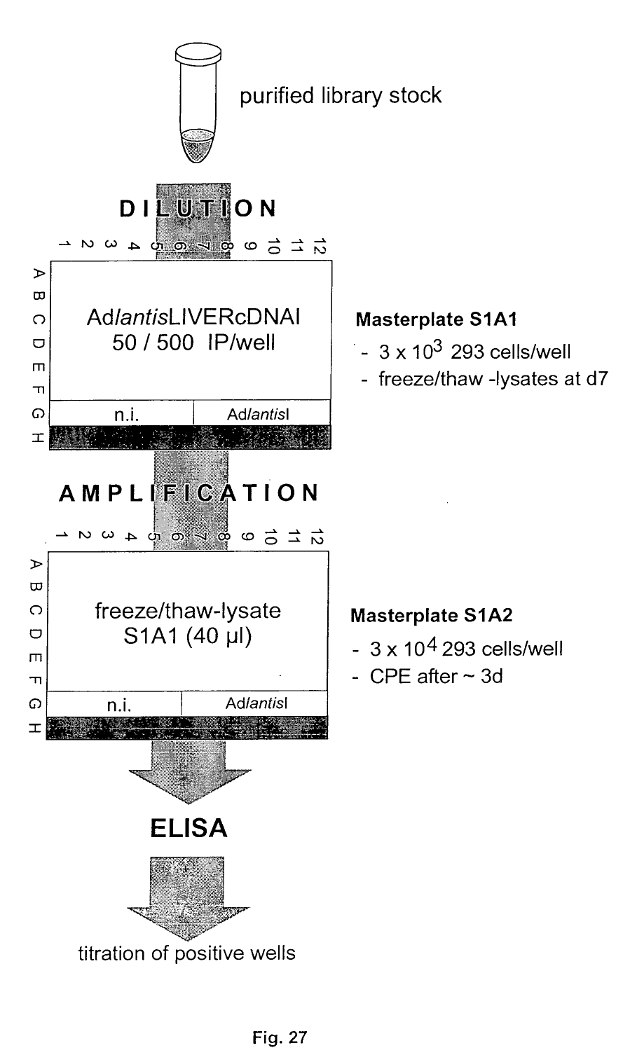

FIG. 27 shows the schematic that was taken as basis for the first screening round of the adenoviral liver cDNA expression libraries (AdlantisLIVERcDNA) for recombinant adenoviruses, which contain the hAAT or hFIX cDNA. For the generation of the masterplates S1A1, 3×10³ 293 cells were seeded into 96 well-plates. Starting from the purified and titrated virus preparations, wells A1-F12 were infected with 50 (first screening round hAAT), or 500 (first screening round hFIX), infectious particles per well. Non-infected cells (wells G1-G6) and cells infected with 50 (first screening round hAAT) or 500 (first screening round hFIX) infectious particles AdlantisI per well (wells G7-G12), served as controls. After 7 days the amplified viruses were set free through freeze/thaw lysis of the cells in the masterplates. In each case, 40 μl of the virus-containing supernatants were used for the infection of 96-well-plates with 3×10⁴ 293 cells per well (masterplates S1A2). After the occurrence of the virus-induced cytopathic effect after approx. 3 days, the cell culture supernatant were tested by means of ELISA for hAAT or hFIX.

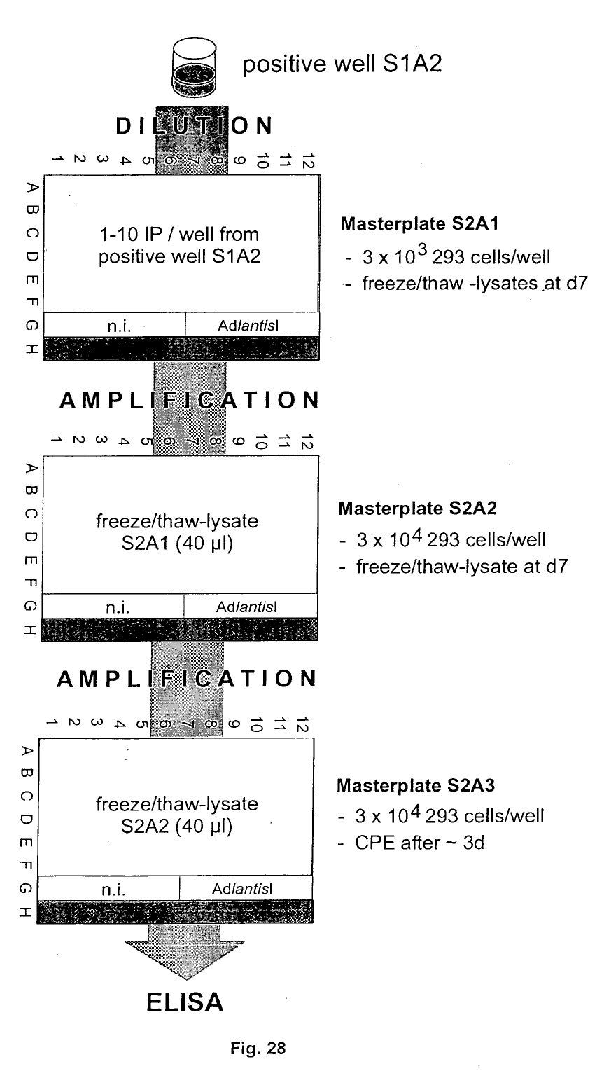

FIG. 28 shows the schematic that was taken as basis for the second screening round of the adenoviral liver cDNA expression libraries (AdlantisLIVERcDNA) for recombinant adenoviruses, which contain the hAAT or hFIX cDNA. For the generation of the masterplates S2A1, 3×10³ 293 cells were seeded into 96 well-plates in each case. Starting from titrated positive wells in S1A2, wells A1-F12 were infected with 1 (second screening round hAAT) and 10 (second screening round hFIX) infectious particles per well. Non-infected cells (wells G1-G6) and cells infected with 1 (second screening round hAAT) or 10 (second screening round hFIX) infectious particles AdlantisI (wells G7-G12), served as controls. After 7 days the amplified viruses were set free through freeze/thaw lysis of the cells in the masterplates. In each case, 40 μl of the virus-containing supernatants were used for the infection of 96-well plates with 3×10⁴ 293 cells per well (masterplates S2A2). With the second screening round for hFIX, after occurrence of the virus induced cytopathic effect (CPE), the cell culture supernatants of these masterplates were tested by means of ELISA for hFIX. In contrary, with the second screening round for hAAT the amplified viruses in S2A2 after 7 days were in set free in the masterplates through freeze/thaw lysis and 40 μl of the virus-containing supernatants used for the infection of 96-well plates with 3×10⁴ 293 cells per well (masterplates S2A3). After occurrence of the CPE, the cell culture supernatants of S2A3 were tested by means of ELISA for hAAT.

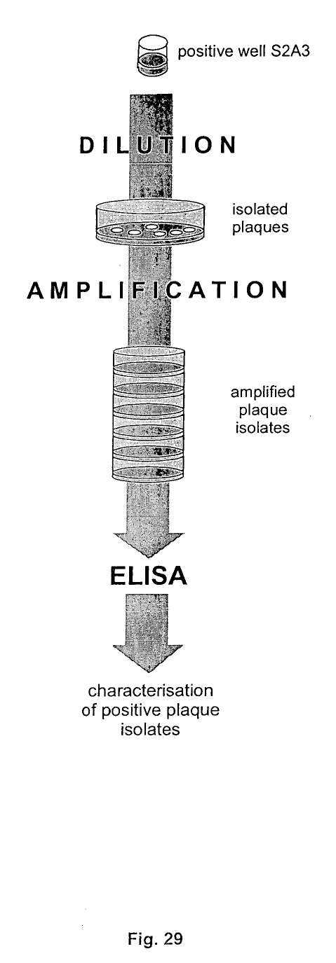

FIG. 29 shows the experimental schematic for the clonal separation of recombinant adenoviruses, which contain the hAAT cDNA or hFIX cDNA, from the positive wells in S2A3 (second screening round hAAT) and S2A2 (second screening round hFIX). Through plaque assays with serial dilutions of the freeze/thaw lysates from the positive wells of the second screening round, separated virus plaques are recovered. The plaque isolates are then amplified individually on 293 cells and the cell culture supernatants are then tested by ELISA for hAAT and hFIX. In case of the positive plaque isolates, the presence of the hAAT cDNA and hFIX cDNA is then verified by sequencing.

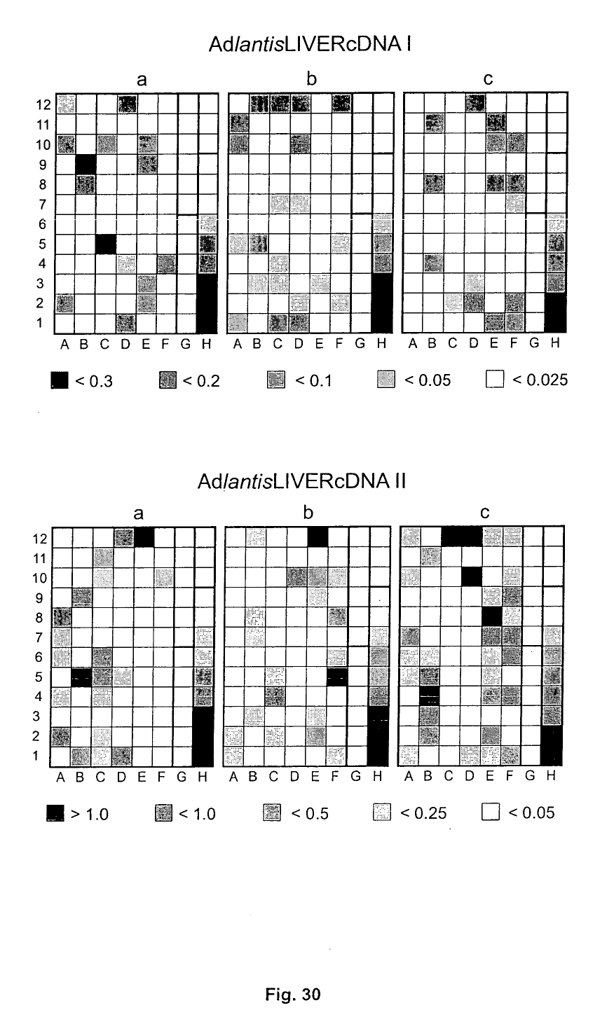

FIG. 30 shows the results of the first screening round of the two adenoviral liver cDNA expression libraries AdlantisLIVERcDNAI (above) and AdlantisLIVERcDNAII (below) for recombinant adenoviruses, which contain the hAAT cDNA. Represented are the raw data of the hAAT-ELISA's (OD₄₉₀) with the supernatants of the 3 (a, b, c) masterplates S1A2 in each case, which, according to the schematic of FIG. 27, were generated with 50 infectious particles/well in A1S1 (A1-F12: Samples; G1-G6: Negative controls 1 (supernatants of non-infected 293 cells; G7-G12: Negative controls 2 (supernatants of cells infected with AdlantisI); F1-F9: Standard series hAAT (1:2 dilution stages starting with 250 ng/μl); F10-F12: blanks).

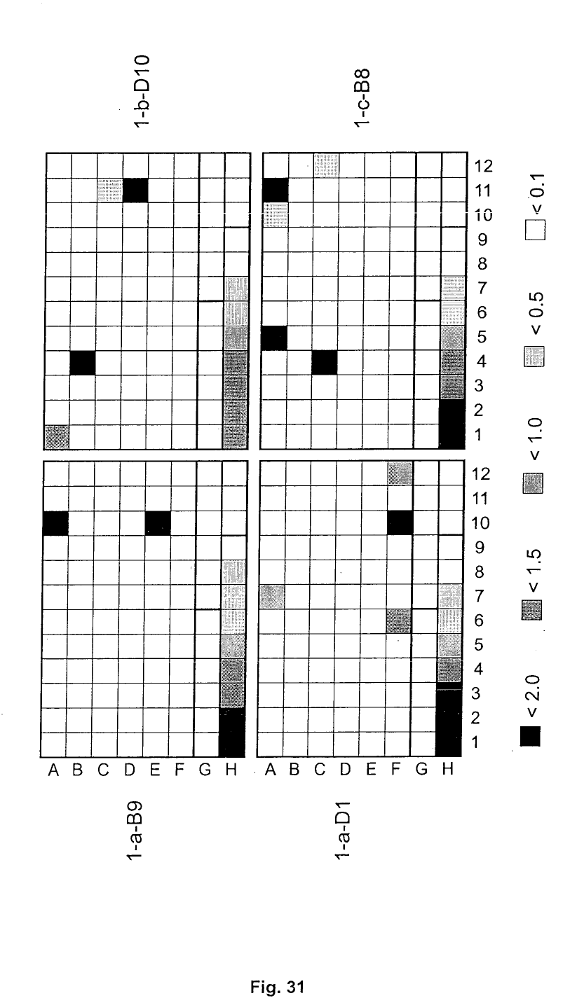

FIG. 31 shows the results of the second screening round of the adenoviral liver cDNA expression library AdlantisLIVERcDNAI for recombinant adenoviruses, which contain the hAAT cDNA. Represented are the raw data of the hAAT-ELISA's (OD₄₉₀) with the supernatants of the 1 masterplate each of S2A3 are represented, which were generated per selected positive subpopulation (1-a-B9,1-a-D1, 1-b-D10 and 1-c-B8) of the masterplates S1A2, according to the schematic of FIG. 28, with 1 infectious particle per well in S2A1 (A1-F12: Samples; G1-G6: Negative controls 1 (Supernatants of non-infected 293 cells; G7-G12: Negative controls 2 (supernatants of cells infected with AdlantisI); F1-F9: Standard series hAAT (1:2 dilution stages, starting with 250 ng hAAT/μl; F10-F12: blanks).

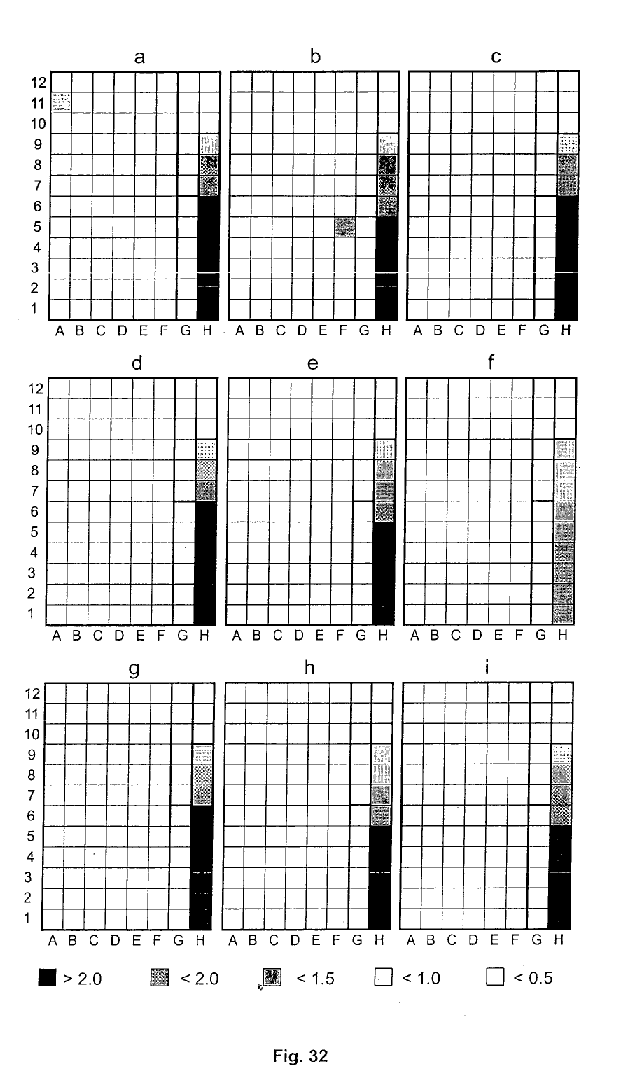

FIG. 32 shows the results of the first screening round of the adenoviral liver cDNA expression library AdlantisLIVERcDNAI for recombinant adenoviruses, which contain the hFIX cDNA. Represented are the raw data of the hFIX-ELISA's (OD₄₉₀) with the supernatants of the 9 (a-f) masterplates S1A2, which were generated according to the schematic of FIG. 27, with 500 infectious particles per well in S1A1 (A1-F12: Samples; G1-G6: Negative controls 1 (Supernatants of non-infected 293 cells; G7-G12: Negative controls 2 (supernatants of cells infected with AdlantisI); E1-F9: Standard series hFIX (1:2 dilution stages starting with 100 ng/μl); F10-F12: blanks).

FIG. 34 shows the results of the second screening round of the adenoviral liver cDNA expression library AdlantisLIVERcDNAI for recombinant adenoviruses, which contain the hFIX cDNA. Represented are the raw data of the hFIX-ELISA's (OD₄₉₀) with the supernatants of the two masterplates (A, B) S2A2, which were generated per selected positive sub-population (I-a-A11 and 1-b-F5) of the masterplates S1A2, according to the schematic of FIG. 28, with 10 infectious particles per well in S2A1 (A1-F12: Samples; G1-G6: Negative controls 1 (supernatants of non-infected 293 cells; G7-G12: Negative controls 2 (supernatants of cells infected with AdlantisI); E1-F9: Standard series hFIX (1:2 dilution stages, starting with 25 ng hFIX/μl); F10-F12: blanks).

### IMPLEMENTATION EXAMPLES

1. System for the Construction of Clonal or Complex Populations of E1-Deleted Recombinant Adenoviruses of the Human Serotype 5

The invention-related system for the generation of rAd was realized for the construction of clonal or complex populations of recombinant E1-deleted human adenoviruses of the serotype 5 (Ad5). The packaging signal of Ad5 consists of seven so-called A repeats, which lie between nt 200 and nt 380 at the 5′-end of the Ad5 genome (Schmid, S. I. and Hearing, P. (1997) J. Virol. 71: 3375-3384). The CrelloxP recombination system of the bacteriophage PI was used as a site-specific recombination system, consisting of the Cre-recombinase and the loxP sequence recognized by it (Sternberg, N. and Hamilton, D. (1981) J. Mol. Biol. 150: 467-486). I-SceI, which has an 18 bp recognition sequence (Monteilhet, C., Rerrin, A., Thierry, A., Colleaux, L. and Dujon, B. (1990) Nucleic Acids Res. 18: 1407-1413) was used as a rare-cutting restriction endonuclease in donor plasmids of the type 2. In the following, the components of the system and their generation are described.

Construction of the Donor Viruses

E1-deleted replication-deficient viruses derived from Ad5 are used as donor viruses, whose packaging signal (i) is partially deleted and (ii) is framed from parallel oriented loxP sequences. Furthermore, the donor viruses have a 2.7 kb deletion in the E3 region and can thus accept up to 8 kb of foreign DNA. There are two donor viruses—AdlantisI and AdlantisII—which are identical in their structure, but however, are distinguished through the extent of the deletion of the packaging signal, (see FIG. 4). In case of AdlantisI, the A repeats VI and VII are deleted, thus it contains the A repeats I-V (nt 194-358 of the Ad5-genome). In case of AdlantisII the A repeats III, IV and V are deleted, thus it contains the A repeats I, II, VI and VII (nt 194-271 and following this nt 355-542 of the Ad5 genome).

The construction donor virus genome was done through homologous recombination in E. coli. First of all, shuttle plasmids were constructed, which contain the 5′-end of the donor viruses (pAd2lis for AdlantisI and pAd2lisΔ for AdlantisII).

Starting plasmid for the construction of pAd2lis was p_E1-2lox, which contains in sequential sequence the 5′ITR of AdS, a loxP sequence, a partially deleted packaging signal of Ad5 with the A repeats I-V (ΔΨIV-VII), a 929 bp non-coding spacer fragment (spacer), a second parallel-oriented loxP sequence and following this the nt 3524-5790 of the Ad5 genome (Hiligenberg, M., Schnieders, F., Löser, P. und Strauss, M. (2001) Hum. Gene Ther. 12: 642-657). The mentioned functional elements were set free from p_E1-2lox as 3008 bp AflIII/BstEII fragment and inserted via the same restriction sites into the shuttle plasmid pHVAd2 (Sandig, V., unpublished), from which pAd2lis arose.

For the construction of pAd2lisΔ, the partially deleted packaging signal ΔΨVI-VII was replaced in pAd2lis by the partially deleted packaging signal ΔΨIII-V. Starting point was the plasmid pSLITRPS, which contains the first 542 bp of the Ad5 genome, including the 5′ITR and the complete packaging signal (Hillgenberg, M., Schnieders, F., Löser, P. und Strauss, M. (2001) Hum. Gene Ther. 12: 642-657). From this, a 704 bp SalI/NruI fragment was cut out, which contains the mentioned Ad5 sequence, and was inserted into the DsaI site of the plasmid pBSSK-(Stratagene), resulting in plasmid pBSITRPS. From this, an 84 bp-DsaI/MluNI fragment was cut out, which corresponds to the nt 272-355 of the Ad5 genome and which contains the A repeats III-V. Through religation of the vector, the plasmid pBSITRPSΔ was obtained, which contains the partially deleted packaging signal ΔΨIII-V. This was then cut out as 249 bp BsrGI/Asp718 fragment and was inserted between the HindIII- and Asp718 sites of pAd2lis, instead of the partially deleted packaging signal ΔΨVI-VII, from which pAd2lisΔ resulted.

The viral 5′-ends to be inserted were set free from pAd2lis and pAd2lisΔ through digestion with Asp700 and StuI and, together with the ClaI-linearized pHVAd1, co-transformed for recombination in E. coli. pHVAd1 (Sandig, V., unpublished) contains the rest of the Ad5 genome with a 2.7 kb deletion in the E3 region. The genomes of the donor viruses AdlantisI and AdlantisII obtained through this recombination were set free from the plasmids pAd1lis and pAd1llisΔ by digestion with PacI, and then transfected into 293 cells. The 293 cells complement the E1-deficiency of the donor viruses, through which a virus amplification can occur. The infectious viruses obtained from this were then further amplified on 293 cells. Atlantis was set free as a supernatant from lysed infected 293 cells and subsequently purified via CsCl density gradients, AdlantisII was used directly as supernatant from lysed infected 293 cells.

Packaging Cell Line

The cell line CIN1004, derived from 293 cells, is used as packaging cell line, which constitutively expresses at high levels the gene for a nuclear-localized Cre-recombinase (Hiligenberg, M., Schnieders, F., Löser, P. und Strauss, M. (2001) Hum. Gene Ther. 12: 642-657). The construction of this cell line had been possible through the employment of a bicistronic vector, where the expression of a nuclear-localized Cre-recombinase was coupled via an internal ribosome entry site with the expression of the selectable neo gene. After transfection of 293 cells with this vector, a direct selection for the high expression of the Cre-recombinase could be done via a selection for high expression of the neo gene.

Construction of the Donor Plasmids

Donor plasmids are used which correspond to donor plasmids of the type 1, 2 and 3 (pCBI, pCBII and pCBIII). They contain one (pCBI, pCBII) or two (pCBIII) loxP recognition sites and the complete packaging signal of Ad5 (A repeats I-VII, nt 194-526 of the Ad5 genome). Furthermore, pCBII in addition contains two recognition sites for the rare cutting restriction endonuclease I-SceI (18 bp identification sequence). The plasmids are present in different forms (see FIG. 5), for example with a polylinker, into which complete expression cassettes can be inserted with promoter, coding region and polyadenylation signal (pCBI-3, pCBII-3, pCBIII-3) or with a polylinker, which is framed by the hCMV promoter and the hCMV polyadenylation signal, for the insertion of coding sequences, for example transgenes or cDNA libraries (pCBI-CMVII, pCBII-CMVII, pCBIII-CMVII).

The donor plasmids were constructed starting from pMV, a plasmid which sequentially contains, besides a bacterial replication origin (ColE1), a cos-signal, and the ampicillin-resistance gene, a recognition site for 1-SceI, nt 1-542 of the Ad5 genome (5′ITR and complete packaging signal), a polylinker, the 3′ITR of Ad5 and a second recognition site for I-SceI (Hillgenberg, M., Schnieders, F., Loser, P. und Strauss, M. (2001) Hum. Gene Ther. 12: 642-657). For the construction of pCBI-3 and pCBI-CMVII, first of all, pMVI was obtained through insertion of a 107 bp XmaI fragment, which contains a loxP recognition site, into the SgrAl site between the Ad5-5′-ITR and the Ad5 packaging signal in pMV. From pMVI, a 905 bp DraI fragment was set free, which contained the 1-SceI identification sequence, the Ad5-5′ITR, the loxP recognition sequence, the Ad5 packaging signal and the polylinker. This was brought to ligation with a 2348 bp PsilPvuII fragment from pBSKS-(Stratagene), which contains a bacterial replication origin (ColE1), the ampicillin-resistance gene and a part of the F1 replication origin, which resulted in pCBI-1.o2. After cutting out the 1-SceI recognition sites and the Ad5 5′ITR as a 311 bp SapI/BamHI fragment and subsequent religation of the vector, pCBI-2 was obtained from pCBI-1.o2. By cutting out of the part of the F1 replication origin as a 284 bp NgoMI fragment and religation of the vector, pCBI-3 was obtained. Through insertion of a 688 bp fragment, which contains the hCMV promoter and the hCMV polyadenylation signal with an intervening polylinker between the PmlI and NaeI site of the polylinker of pCBI-3, pCBI-CMV was obtained. Through insertion of a linker, which was obtained through hybridization of the oligonucleotides 5′-AATTGTTTAAACGGCCCTCGAGCCGT-3′ and 5-ATACGGCCTCGAGGGCCGTTTAAAC-3′, between the MunI and AccI sites of the polylinker of pCBI-MV, pCBI-CMVII was obtained.

For the construction of pCBII-3 and pCBII-CMVII, the Ad5-3′ITR was excised from pMV as 261 bp BglII fragment, the religation of the vector resulted in pCBII-1. Through insertion of a 107 bp fragment with a loxP recognition sequence into the polylinker of pCBII-1, pCBII-2 was obtained. After cutting out the cos-sequence as 2332 bp EcoNI/SapI fragment from pCBII-2, pCBII-3 was obtained. Through insertion of a 688 bp fragment, which contains the hCMV promoter and the hCMV polyadenylation signal with an intervening polylinker, between the PmlI and Bst1107i sites of the polylinker of pCBII-3, pCBII-CMV was recovered. Through insertion of a linker, which was obtained through hybridization of the oligonucleotides 5′-AATTGTTTAAACGGCCCTCGAGGCCGT-3′ and 5-ATACGGCCTCGAGGGCCGTTTAAAC-3′, between the MunI and AccI sites of the polylinker of pCBII-CMV, pCBII-CMVII was obtained.

pCBIII-3 and pCBIII-CMVII were constructed starting from pCBI-3 (see above). First of all, the plasmid pCBIII-3 was obtained through insertion of a 107 bp fragment with a loxP-sequence into the NgoMI site of the polylinker of pCBI-3. Through insertion of a 688 bp fragment, which contains the hCMV promoter and the hCMV polyadenylation signal with an intervening polylinker, into the Bst1107i site of the polylinker of pCBIII-3, pCBIII-CMV was obtained. Through insertion of a linker, which was obtained through hybridization of the oligonucleotides 5′-AATTGTTTAAACGGCCCTCGAGGCCGT-3′ and 5-ATACGGCCTCGAGGGCCGTTTAAAC-3′, between the MunI and AccI sites of the polylinker pCBIII-CMV, pCBIII-CMVII* was constructed. Through cutting out of a 43 bp PmlI/NruI fragment from pCBIII-CMVII*, which containd a XhoI site in addition to the one present in the polylinker, followed by the religation of the vector, pCBIII-CMVII was obtained.

Functional Testing of the Donor Viruses

For the testing of the efficiency of the generation of the donor virus ΔΨ acceptor substrate, CIN1004 cells were infected with AdlantisI or AdlantisII. After occurrence of the virus-induced cytopathic effect, the replicated viral DNA was isolated and subjected to a restriction analysis, by which unprocessed donor virus and processed donor virus ΔΨ acceptor substrate can be distinguished. The fragment pattern corresponded completely to processed donor virus ΔΨ acceptor substrate (FIG. 4B). Furthermore, the comparison of the number of infectious particles produced per cell as progeny after infection of CIN1004 or 293 cells indicated an approx. 100× growth reduction of the donor viruses of AdlantisI and AdlantisII on CIN1004 cells (FIG. 4C), as result of the excision of the viral packaging signal. These findings indicate a very high efficiency of the formation of the donor virus ΔΨ acceptor substrate. Furthermore, also the growth of AdlantisI and AdlantisII on 293 cells (FIG. 4C) was compared in these experiments. It was shown that AdlantisII is more severely packaging inhibited by about 100× in comparison with AdlantisI on both cells lines, a result of the packaging signal deleted more extensively in comparison with AdlantisI.

Functional Testing of the Donor Plasmids

For the testing of the rescue of rAd after transfection of donor plasmids into the donor virus-infected packaging cell line, a constitutive expression cassette for the reporter gene DsRed was inserted into the polylinker of the donor plasmids pCBI, pCBII and pCBIII. The donor plasmids pCBI-DsRed, pCBII-DsRed and pCBIII-DsRed thus obtained, as well as the resulting recombinant adenoviruses AdCBI-DsRed, AdCB1I-DsRed and AdCBIII-DsRed from these donor plasmids through CrelloxP-mediated recombination with the donor virus ΔΨ acceptor substrate, are shown in FIG. 6. For the construction of these viruses pCBI-DsRed, pCBII-DsRed (digested with I-SceI) and pCBIII-DsRed were transfected into CIN1004 cells, which had been infected before with AdlantisI. After occurrence of the virus-induced cytopathic effects (CPE) the cells were lysed (freeze/thaw lysate amplification round 0, A0). The virus-containing lysate thus obtained was used, for the purpose of amplification of the recombinant adenoviruses, for the infection of 293 cells, which were lysed in turn after the occurrence of the CPE (freeze/thaw lysate amplification round 1, A1). Then the total amount of the recombinant adenoviruses AdCBI-DsRed, AdCBII-DsRed, and AdCBIII-DsRed contained in A0 and A1 were determined. The detection was done via the DsRed reporter gene cassette by means of fluorescence microscopy as DsRed transducing units (DTU) after infection of cells. Furthermore, the total amount of infectious particles in A0 and A1 was titrated through dilution end point analysis on 293 cells (FIG. 7). With use of all three donor plasmids, DTU could be detected, thus recombinant adenoviruses had formed. The total amount at DTU was approx. 100 in A0 and approx. 1000 in A1, with employment of the donor plasmids pCBI-DsRed and pCBIII-DsRed. In contrary, the total amount of infectious particles was approx. 10⁵ in A0 and approx. 10⁷ in A1, which indicated a strong contamination of the recombinant adenoviruses with residual donor virus. With employment of I-SceI-digested pCBII-DsRed as a donor plasmid, on the other hand, the total amount of DTU, with approx. 10⁵ in A0 and approx. 10⁷ in A1, was far higher, with a comparable total amount of infectious particles.

In order to characterize the virus mixtures more precisely, 293 cells were infected with the freeze/thaw lysates A1. After occurrence of the virus-induced cytopathic effect, the replicated viral DNA was extracted & analyzed by means of digestion with PshAI (FIG. 8A). This enzyme generates, for the recombinant adenoviruses AdCBI-DsRed, AdCBII-DsRed and AdCBIII-DsRed, as well as for the donor virus AdlantisI, in each case a characteristic 5′-terminal fragment (cf. FIG. 6). In the digestions of the Hirt extracts, which had been obtained after employment of pCBI-DsRed and pCBIII-DsRed as donor plasmids, only the 5′-terminal fragment of AdlantisI could be detected. In the digestions of the Hirt extracts, which had been obtained after employment of pCBII-DsRed as a donor plasmid, in contrary, both 5′-terminal fragments characteristic for AdlantisI, as well as for the recombinant adenovirus AdCBII-DsRed, could be detected in a ratio of approx. 1:1. These results confirm the almost equally high total amounts of DTU and infectious particles shown in FIG. 7 in A1 with employment of pCBII-DsRed as a donor plasmid. Furthermore, they indicate that the far higher total number of infectious particles with employment of pCBI-DsRed and pCBIII-DsRed as donor plasmid, in relation to the total amount of DTU, reflects a strong contamination with residual donor virus.

In order to further prove the rescue of the recombinant adenoviruses, PCR analyses were carried out with the same Hirt extracts, with which primer pairs were used, that give rise to a product only from the recombinant adenoviruses AdCBI-DsRed, AdCBII-DsRed and AdCBIII-DsRed, but from the donor virus or the donor plasmids (cf. FIG. 6). In all experiments, products characteristic for the recombinant adenoviruses were generated (FIG. 8B). This proves that the DTU in A0 and A1 in FIG. 7 actually correspond to the recombinant adenoviruses, and that the absence of the 5′-terminal fragment characteristic for AdCBI-DsRed and/or AdCBIII-DsRed, with employment of the donor plasmids, was caused only through the high contamination with residual donor virus.

The testing of the donor plasmids thus gave the result that recombinant adenoviruses in reproducible form arise with employment of all three donor plasmid types 1 2 and 3, and that, with employment of type 2 donor plasmids (pCBII-DsRed), the efficiency of the rescue of the recombinant adenoviruses is most efficient and, furthermore, the contamination with residual donor virus is lowest.

Therefore, as a result, for the generation of clonal and complex populations of recombinant adenoviruses, type 2 donor plasmids were used in the following (derivatives of pCBII-3 or pCBII-CMVII, cf. FIG. 5).

2. Construction of Clonal rAd Populations

For the generation of clonal populations of rAd, the donor plasmids pCBII-DsRed and pCBII-lacZ were used (type 2 donor plasmids), which as transgenes contain RSV promoter-driven constitutive expression cassettes for the reporter genes DsRed and lacZ. Similar to pCBII-DsRed (see above), pCBII-lacZ was obtained through insertion of the expression cassette into the polylinker of pCBII-3. Both plasmids, as well as the recombinant adenoviruses AdCBII-DsRed and AdCBII-lacZ arising from recombination with the donor virus ΔΨ acceptor substrate, are shown in FIG. 9. The efficient formation of recombinant adenoviruses from donor plasmids of the type 2, in association with AdlantisI as a donor virus, had been shown already in the previous experiments. In the following, it was tested under which conditions large scale preparations of clonal recombinant adenoviruses can be obtained with sufficient purity. In particular, it was the goal to obtain (1) minimal contamination with residual donor virus, (2) structural integrity of the recombinant adenoviruses during amplification and (3) absence of contamination with replication-competent wild type adenovirus (RCA), which, as is generally known, frequently arises during the amplification of recombinant adenoviruses on 293 cells.

The invention-related system for the generation of recombinant adenoviruses enables, with employment of type 2 donor plasmids, the utilization of two, where appropriate also combinable, selection principles for the reduction of the contamination by residual donor virus: (1) The donor viruses contain, unlike the recombinant adenoviruses, a deletion in the packaging signal. Through the direct competition for preformed capsids in the infected cells, the recombinant adenoviruses should have a growth advantage as a result. This advantage should stand in reverse relationship to the scale of the deletion of the packaging signal, which is different in the donor viruses AdlantisI and AdlantisII. In order to test the efficiency of this selection principle, it was necessary to determine how high the contamination is in large scale preparations of clonal recombinant adenoviruses, with employment of both donor viruses after amplification on normal 293 cells. (2) In case of amplification of the recombinant adenoviruses on the Cre-recombinase expressing packaging cell line CIN1004, an additional selection principle is active: Recombinant adenoviruses, which arise with employment of donor plasmids of the type 2, contain only one loxP-recognition site. In CIN1004 cells they are thus not a substrate for the CrelloxP-provided excision of the packaging signal, unlike the donor viruses, whose packaging signal is framed by two loxP-sequences, and whose growth on CIN1004 cells is thereby reduced approx. 100× (cf. FIG. 4).

Thus it was initially the objective to determine the residual donor virus contamination in clonal populations of recombinant adenoviruses, which had been amplified on 293 cells (selection only via the partially deleted packaging signal) or amplified on CIN1004 cells (selection via the excision of the donor virus packaging signal and via the partially deleted packaging signal). FIG. 10 summarizes the experimental procedure. After the infection of 10⁶ CIN1004 cells with AdlantisI or AdlantisII in each case, the cells were transfected with the I-SceI-digested donor plasmids pCBII-DsRed or pCBII-lacZ. After occurrence of the virus-induced cytopathic effect, the cells were lysed. In each case ⅕ ml of the thus obtained virus-containing lysate was used for the amplification on 293 or CIN1004 cells, where a 60 mm dish (Amplification round 1, A1), a 150 mm dish (amplification round 2, A2) and finally 10 15 mm dishes (amplification round 3, A3) were used sequentially. The viruses set free from the last amplification round were purified using CsCl density gradient centrifugation and after separation of the CsCl through gel filtration, 2 ml each of purified virus preparation were obtained. Three independent parallel experiments were done in each case for both donor plasmids pCBII-DsRed and pCBII-lacZ, in combination with the two donor viruses AdlantisI and AdlantisII, as well as with amplification on 293 cells and on CIN1004 cells and therefore a total of 24 large scale preparations were obtained.

In order to check the ratio of recombinant adenoviruses and residual donor viruses after the first amplification round (A1), 293 cells were infected with ⅕ of the freeze/thaw lysate from A1, and then the replicated viral DNA was isolated and and analyzed by restriction digestion with PshAI (FIG. 11), which generates characteristic 5′-terminal fragments from the recombinant adenoviruses AdCBII-DsRed and AdCBII-lacZ, as well as from the donor viruses AdlantisI and AdlantisII (cf. FIG. 9). In all approaches, the existence of the recombinant adenoviruses was identified by means of the presence of the characteristic 5′-terminal fragments. With the employment of AdlantisI as donor virus and amplification on 293 cells, the 5′-terminal fragment of the donor virus could be additionally identified in all experiments, which indicated a residual contamination with this donor virus. In contrary, with employment of AdlantisII as donor virus and amplification on 293 cells, the 5′-terminal fragment of the donor virus did not occur. This indicates an increased reduction of the donor virus contamination through the packaging signal of AdlantisII, deleted more strongly in comparison with AdlantisI. In case of amplification on CIN1004 cells, with employment of both donor viruses the 5′-terminal fragment of the donor virus could not be detected. This indicates an increased reduction of the donor virus contamination through the Cre-loxP-mediated excision of the donor virus packaging signal in these cells.

For the analysis of the mixture ratios of recombinant adenoviruses and residual donor viruses, as well as for the verification of the structural integrity of the recombinant adenoviruses after the amplification, viral DNA was extracted from the purified virus preparations and analyzed by digestion with PshAI. In all purified virus DNAs, only the characteristic 5′-terminal fragment of the recombinant adenovirus could be detected (FIG. 12). Contamination with residual donor virus could thus not be detected with this method. Furthermore, these results proved that the structure of the recombinant viruses had remained intact during the amplification.

The titer of intact infectious particles (through dilution end-point analysis on 293 cells) and the total titer of viral particles (through measurement of the photometric absorption of the virus-preparation) were then determined for all purified large scale preparations of AdCBII-DsRed and AdCBII-lacZ (FIG. 13). Both the titers of intact infectious particles (10¹⁰-10¹¹ per ml), as well as the ratios of the total titer of viral particles to the titer on infectious particles (15-43), lie within the range of the values which are also obtained with other usual methods for the generation and amplification of recombinant adenoviruses.

Since restriction analysis of viral DNA isolated from purified virus is not sensitive enough for the detection of low-level contamination with residual donor virus, Southern Blot analyses were carried out for more precise quantification of contamination. Virus DNA, isolated from the purified virus preparations and digested with PshAI, as well as a probe which specifically binds to the 5′-end of the donor viruses, were used. Serial dilutions of donor virus DNA (FIG. 14) served as controls. Through comparison of the intensity of the signals with the controls the following contamination with residual donor virus was determined: In case of utilization of AdlantisI as a donor virus and amplification on 293 cells, the contamination is about 1%. This is a result of the selection against the donor virus with partially deleted packaging signal during the amplification of the recombinant adenoviruses, since the donor virus contamination in A1 was still at about 50%. In case of utilization of AdlantisI as a donor virus and amplification on 293 cells, the contamination was only at approx. 0.03%. This is a result of the reinforced selection for the recombinant adenoviruses with employment of AdlantisII as a donor virus, through its more extensively deleted packaging signal. In case of amplification on CIN1004 cells, no signal could be identified with employment of both donor viruses, the contamination was thus under the last dilution stage of the donor virus DNA, which still supplied a signal in the control, thus under 0.001%. Through combination of both selection principles by means of amplification of the recombinant viruses on CIN1004 cells, large scale preparations can thus be obtained, whose purity is sufficient, with respect to the donor virus residual contamination, for most applications of recombinant adenoviruses.

All purified large scale preparations of AdCBII-DsRed and AdCBII-lacZ were then tested for contamination with replication-competent wild type adenoviruses (RCA). These arise, as is generally known, with a frequency which cannot be neglected in the amplification of recombinant adenoviruses on 293 cells. This is caused by homologous recombination events between the 5′-termini of the recombinant adenoviruses and the 4344 5′-terminal bp of Ad5 inserted into the genome of 293 cells (and also the CIN1004 cells derived from them). Wild type viruses arise from a double crossover event and have no E1 deficiency. For the testing of the preparations, 10⁸ infectious particles were used in each case for the infection of Huh7 cells, on which only RCA are enabled for replication. After 7 days the cells were lysed and ⅓ of the lysate was used for the infection of Huh7 cells, for further amplification of possibly formed RCA. After a further 7 days, the cell culture supernatants were tested by means of PCR for the presence of RCA (FIG. 15). In 12/12 preparations, which had been obtained with AdlantisI as a donor virus, contamination with RCA could be found. In contrary, an RCA contamination was found only in 1/12 preparations when AdlantisII had served as a donor virus. A RCA contamination in 1/12 preparations is within the range of what is also observed with previous conventional methods for the generation and amplification of recombinant adenoviruses.

Since the recombinant adenoviruses AdCBII-DsRed and AdCBII-lacZ are identical with utilization of both donor viruses, it was unlikely that the RCA arose from these recombinant adenoviruses during their amplification. It rather had to be assumed that they arose, with a high degree of probability, after the infection of CIN1004 cells in A0 specifically with employment of AdlantisI and then grew during the amplification of the recombinant adenoviruses. In order to verify this, AdlantisI and AdlantisII were passaged once through 293 cells and CIN1004 cells. The same conditions were applied as in case of the A0 for the generation of recombinant adenoviruses according to the schematic of FIG. 10: Infection of 60 mm dishes with 5 infectious particles per cell of AdlantisI and with 1 infectious particle per cell of AdlantisII. After occurrence of the virus-induced cytopathic effect, the cells were lysed through freeze/thaw lysis and the lysates were used in a PCR to detect RCA (FIG. 16). While, in the case of AdlantisII after passage through 293 or CIN1004 cells, no RCA were found in any of the four parallel experiments each, RCA occurred in the case of AdlantisI after passage through CIN1004 cells in 3/4 of the experiments, but not after passage through 293 cells. It can thus be excluded that the RCA were already containd in the preparation of AdlantisI. Rather they must have newly arisen after infection of CIN1004 cells.

In conclusion, it can be summarized that with employment of AdlantisII as a donor virus, in association with donor plasmids of the type 2 (pCBII-3 derivatives), large scale preparations can reproducibly be obtained by direct amplification on 293 or CIN1004 cells of recombinant adenoviruses generated in A0, which (1) contain the recombinant adenoviruses with intact genome structure at high titers, (2) contain a residual donor virus contamination of less than 0.001% and (3) are not contaminated with RCA. In total, the invention-related process, in contrast to previous processes for the generation of clonal populations of adenoviruses, represents progress, since it requires far less work stages and furthermore is simpler and faster regarding handling. Furthermore, it is cheaper with regard to the costs of materials.

3. Construction of Complex rAd Populations

For the determination of the number of independent rAd formation events, which is a measure of the complexity to be achieved with the system during the generation of mixed rAd populations, AdlantisI and AdlantisII were used as donor viruses together with mixtures of the donor plasmids pCBII-DsRed and pCBII-lacZ (see above). After the infection with AdlantisI (5 infectious particles per cell) or AdlantisII (1 infectious particle per cell), in each case 10⁶ CIN1004 cells were transfected with 12 μg each of different mixtures of I-SceI-digested pCBII-DsRed and pCBII-lacZ. Molar mixture ratios from 50:1 to 500,000:1 were used. After occurrence of the cytopathic effect, the cells were lysed and the viruses contained in the lysate were amplified once on 293 cells. Then the total amount of amplified rAd, which contains the lacZ gene (AdCBII-lacZ), was determined. The detection and titration of the rAd was done via the detection of lacZ reporter gene expression after infection of Huh7 cells (FIG. 17). With employment of AdlantisI as a donor virus, the formation of AdCBII-lacZ was detected at mixture ratios 1:50, 1:500 and 1:5,000 in all cases, and at 1:50,000 in 7/8 cases. With higher mixture ratios AdCBII-lacZ was detected in few experiments only. In case of AdlantisII, even with a dilution of 1:5,000, only 3/9 experiments were positive. The number of independently formed recombinant adenovirus clones per 10⁶ CIN1004 cells is thus approx. 50,000 with the employment of AdlantisI, and far lower with the employment of AdlantisII, between 500-5,000.

The complexity of 50,000 independent clones per 10⁶ cells achieved with AdlantisI means, with employment of only 2×10⁷ cells (corresponds to 20 subconfluent 60 mm dishes), a total complexity of 10⁶ independent clones, which is sufficient for the construction of gene libraries, for example adenoviral cDNA expression libraries. AdlantisII, on the other hand, is unsuitable as a donor virus for the construction of cDNA expression libraries, since a complexity of 10⁶ independent clones would require the employment of 10⁸-10⁹ CIN1004 cells (corresponds to 200-2000 subconfluent 60 mm dishes). In addition, this would require the transfection of a total of 2.4-24 mg cDNA expression library in the donor plasmid. The amplification of cDNA expression libraries in plasmids at such quantities is not possible without loss of complexity.

The schematic in FIG. 18 shows the experimental procedure for the construction of adenoviral expression libraries, as well as the method for the isolation of cDNAs from these through a biological test system. First of all, cDNA is synthesized starting from the Poly-A(+)-RNA from the selected tissue or cell type, and is then inserted directionally between the CMV promoter and the CMV polyadenylation signal, into the polylinker of the donor plasmid pCBII-CMVII. From that a cDNA expression library is obtained in pCBII-CMVII. For the generation of the adenoviral cDNA expression library with 10⁶ independent clones, a total of 20 60 mm cell culture dishes with 10⁶ CIN1004 cells each, are then infected with 5 infectious particles AdlantisI per cell and transfected with 12 μg each of 1-SceI-digested plasmid library. Through amplification of the viruses generated thereby and a subsequent purification of the viruses, a high-titer purified adenoviral cDNA expression library is obtained. In order to isolate adenovirus clones from that by means of a biological test system, which contain cDNAs with certain properties detectable in a biological test system, so-called masterplates are generated through infection of 293 cells in multiwell plates. Here, one or several infectious particles from the adenoviral cDNA expression library are used per well, by which defined monoclonal or oligoclonal sub-populations are amplified. When the cells in the masterplates are completely infected, they are lysed through freeze/thaw lysis. Due to the stability of adenoviruses the masterplates can be stored for a long time by freezing. The supernatants in the wells of the masterplates contain the amplified infectious adenoviruses. Furthermore, they contain the proteins which are coded by the cDNAs contained in the respective adenovirus clones, since the CMV promoter leads to their expression in the infected 293 cells. A direct verification of a protein searched for in the lysates can thus serve as a test system, for example an enzyme-linked immunosorbant assay (ELISA). Or the lysates are used for the infection of cells in a cell-based test system, with which a phenotypic change caused by the expression of the cDNA in the cells can be detected. From those wells of the masterplates, whose supernatants induce the signal searched for in such test systems, the recombinant adenoviruses can be clonally separated out by plaque assay on 293 cells. Then, the cDNAs can be characterized, for example by sequencing.

In order to show that this experimental procedure is in fact possible (proof of concept), an adenoviral cDNA expression library was constructed starting from human liver mRNA. Adenovirus clones were then isolated from it, which contain the cDNAs of the human alpha-1-antitrypsin (hAAT) and the human blood-clotting factor IX (hFIX). ELISAs served as system for the detection of these secreted proteins in the supernatants of the masterplates. These serum proteins, expressed in the liver, were selected because they are a good example for a gene strongly expressed in the liver (hAAT, serum concentration approx. 2 g/l) and a gene weakly expressed in the liver (hFIX, serum concentration approx. 4 mg/l).

Construction of Adenoviral Liver cDNA Expression Libraries

First of all, the expression library was constructed for human liver cDNA in the donor plasmid pCBII-CMVII. The experimental procedure is summarized in FIG. 19. From 5 μg Poly-A(+)-RNA from healthy human liver, cDNA with cohesive EcoRI and XhoI ends was generated with “cDNA synthesis kit” (Stratagene) and, after size fractionating, directionally inserted into the compatible MunI and XhoI restriction sites of the polylinker of pCBII-CMVII. A total of 12 ligations with in each case 30 μg of MunI/XhoI-digested and dephosphorylized vector pCBII-CMVII and 10 ng of cDNA were carried out, and the ligation products were completely transformed into E. coli XL1OGOLD (Stratagene). 8.23×10⁵ transformants were obtained on 24 150 mm agar plates. These were scraped from the plates and amplified in a total of 2 l LB medium. Then, through plasmid extraction and purification, a total of 1800 μg of purified plasmid library pCBII-CMVII-LIVERcDNA was obtained. The purified plasmid library was then characterized concerning the size of the inserted cDNAs. To this end, plasmid DNA from isolated clones was subjected to a restriction analysis with SnaBI. This enzyme cuts out the entire expression cassette, along with CMV promoter, cDNA and polyadenylation signal.

From the size of the fragments, the size of the inserted cDNA can be estimated (FIG. 20A). This was in total 17 clones tested in the range of 400-3100 bp, with an average of approx. 1500 bp. The sizes of the cDNA for hAAT (1258 bp) and hFIX (1390 bp) lie within this range. The presence of the cDNA for hAAT and hFIX in the plasmid library was then confirmed by PCR analyses with primers, which only generate a product when the complete cDNAs are present (FIG. 20B). Thus, a liver cDNA expression library in pCBII-CMVII was constructed, which has a complexity of 8.2×10⁵ independent clones and, as has been proven, contains the cDNAs for hAAT and hFIX.

This plasmid library was then used for the generation of adenoviral liver cDNA expression libraries. The experimental procedure is summarized in FIG. 21. Twenty 60 mm cell culture dishes with 10⁶CIN1004 cells each were infected with 5 infectious particles AdlantisI per cell and then transfected with 12 μg of I-SceI-digested plasmid library pCBII-CMVII-LIVERcDNA per dish. After occurrence of the virus-induced cytopathic effect (CPE), the cells were lysed through freeze/thaw lysis and the lysates from the twenty dishes were pooled (primary adenoviral liver cDNA expression library, amplification round 0, A0). For the amplification of the expression library, the half of the lysate of A0 was used for the infection of four subconfluent 50 mm cell culture dishes with CIN1004 cells. After occurrence of the CPE, the cells were lysed through freeze/thaw lysis (amplification round 1, A1). The half of the lysate of A1 were then used for the infection of nine subconfluent 150 mm cell culture dishes with 293 cells. After occurrence of the CPE, the cells were sedimented and lysed through freeze/thaw lysis (amplification round 2, A2). The viruses thus set free were purified using CsCl density gradient centrifugation and after removal of CsCl two ml of purified adenoviral liver cDNA expression library were obtained. In order to check the conditions of the formation of the primary adenoviral liver cDNA expression library in A0, two types of controls were carried out in parallel: For control of efficiency of virus rescue, three subconfluent 60 mm dishes with CIN1004 cells were transfected with 12 μg I-SceI-digested pCBII-lacZ per dish each after infection with 5 infectious particles AdlantisI per cell. For control of complexity, three subconfluent 60 mm dishes with CIN1004 cells were transfected with 12 μg per dish each of a molar 1:50,000 mixture of I-SceI-digested pCBII-lacZ and I-SceI-digested plasmid library pCBII-CMVII-LIVERcDNA after infection with 5 infectious particles AdlantisI per cell. After occurrence of the CPE, the cells were separately lysed through freeze/thaw lysis (amplification round 0, A0 of the controls). In each case ⅕ of the lysates were then amplified once through 106 293 cells (amplification round 1, A1 of the controls). In each case ⅕ of the lysates of the A1 of the controls were then used for the infection of subconfluent Huh7 cells in 60 mm dishes. After two days, the cells were stained with X-Gal. Through this, blue cells showed an infection from recombinant adenovirus AdCBII-lacZ formed from pCBII-lacZ.

The entire procedure, including controls, was carried out twice independently, which led to the two adenoviral expression libraries AdlantisLIVERcDNAI and AdlantisLIVERcDNAII. The corresponding controls for the efficiency of virus rescue and the complexity of virus rescue are shown in FIG. 22. In all of the three controls each for efficiency, about 50% of the cells stained blue. This was more blue-stained cells than were obtained in parallel experiments after infection of subconfluent Huh7 cells in 60 mm dishes with in total 10⁷ infectious particles AdRSV-lacZ (a recombinant adenovirus with an RSV promoter-driven lacZ expression cassette). The efficiency of the virus rescue in A0 was thus very high. In all of the three controls each for complexity, blue staining cells could be detected, which indicated that in every dish at least one recombinant adenovirus had formed from the 1:50,000-diluted pCBII-lacZ. The complexity in A0 was thus at least 50,000 independent clones per 60 mm dish. With the virus rescue for the generation of the primary adenoviral expression libraries from twenty 60 mm dishes in A0, a complexity of at least 10⁶ independent adenovirus clones could thus be assumed. In conclusion, in two independent experiments, the plasmid library pCBII-CMVII-LIVERcDNA had been converted with high efficiency into adenoviral liver cDNA expression libraries with a complexity of at least 10⁶ independent adenovirus clones.

Characterization of the Adenoviral Liver cDNA Expression Libraries

The titer on intact infectious particles (through dilution end-point analysis on 293 cells) and the total titer of viral particles (through measurement of the photometric absorption of the virus preparation) were then determined for both purified adenoviral cDNA expression libraries. Both the titers on intact infectious particles (in each case 2.3×10¹¹ infectious particles per ml with AdlantisLIVERcDNAI and AdlantisLIVERcDNAII), as well as the ratios of the total titer of viral particles versus the titer of infectious particles (˜10 in case of AdlantisLIVERcDNAI and ˜12 in case of AdlantisLIVERcDNAII) were within the range of what is also achieved with the generation of clonal adenovirus populations with the invention-related system (see above).

Individual clones of recombinant adenoviruses from the two purified adenoviral expression libraries were then obtained by plaque assay on 293 cells, for the characterization of the insert size range. 293 cells were infected with the plaque isolates and following this the replicated viral DNA was isolated and subjected to a restriction analysis with PshAI. This enzyme generates characteristic fragments of the 5′-ends of the recombinant adenoviruses, from whose size the size of the inserted cDNAs can be estimated. Analysis of the 17 plaque isolates gave the results that (1)—identifiable by different fragment sizes—the inserted cDNAs were all different, (2) the sizes of the cDNAs were within the range 300-2700 bp (AdlantisLIVERcDNA I) and 400-2100 bp (AdlantisLIVERcDNAII) and (3) the average size of the cDNAs was about 1300 bp (AdlantisLIVERcDNA 1) and 1500 bp (AdlantisLIVERcDNAII) (FIG. 23). Both the insert size range, as well as the average insert size, agree well with those of the plasmid library pCBII-CMVII-LIVERcDNA (see above), which indicates that, with the conversion into the adenoviral expression libraries and with their amplification, a shift in insert sizes did not occur. The plasmid library pCBII-CMVII-LIVERcDNA had thus been converted into an adenoviral expression library in two independent experiments, with retention of the complexity and the insert size distribution.

Since the use of AdlantisI as donor virus is associated with the danger of contamination of the virus preparations with replication-competent wild type adenovirus (RCA), the extent of contamination was determined for AdlantisLIVERcDNAI and AdlantisLIVERcDNAII. As result a contamination of <1% with AdlantisLIVERcDNAI and about 10% with AdlantisLIVERcDNAII was found (FIG. 24). Due to the smaller contamination with RCA, all the following experiments were carried out with AdlantisLIVERcDNAI.

First of all, it was a matter of being able to characterize individual plaque isolate from AdlantisLIVERcDNAI concerning the inserted cDNAs, in order to make a statement about the percentage content of full-lengh cDNAs in the library. Here, the Hirt extracts of the plaque isolate I-6, I-8, I-11, I-15, I-17, I-18, I-19, I-24, I-25, I-26 and I-28, which had already been used for the restriction analysis with PshAI (see above), were used as substrate in a PCR with primers, which bind in sense-orientation in the CMV promoter (ACCGTCAGATCGCCTGGAGA) and in antisense-orientation in the CMV polyadenylation signal (CGCTGCTAACGCTGCAAGAG). The PCR products were then cloned into the polylinker of pBSKS. With primers, which bind to the T3 and T7 promoters in the plasmid vector located on both sides of the PCR product insertion point, the inserts were then sequenced. By means of BLASTN (www.ncbi.gov), the sequences were compared with sequence databases. The results are combined in tabular form in FIGS. 25 and 26. For 9 of the 11 plaque isolates, the cDNAs were identified. In case of two of the inserts (plaque isolate I-15 and I-19) no agreements with known cDNAs could be found, except for homologies to chromosomal regions. They thus represent genes possibly not characterized up to now. In case of the residual nine inserts, there were five complete cDNAs (I-6, I-8, I-11, I-17, I-26) and four 5′-truncated cDNAs (I-18, I-24, I-25, I-28). The cDNAs coded in six cases for serum proteins, which are synthesized in the liver (apolipoprotein A, complement component 4 binding protein, histidine-rich glycoprotein, vitronectin, 2× haptoglobin) and in three cases for intracellular proteins of the liver (deoxyguanosin kinase, Cytochrom P450, and proteasomal modulator subunit PSMD9). In conclusion, the characterization of the inserts of the plaque isolates from AdlantisLIVERcDNAI gave the result that more than 50% of the cDNA's were full-length, that 2/11 inserts correspond to genes possibly not characterized up to now and that all unambiguously identifiable cDNAs, according to the expectation, code for genes expressed in liver.

Screening of the Adenoviral Liver cDNA Expression Libraries

Sandwich ELISA's with the supernatants of cells, which had been infected with sub-populations from the adenoviral liver cDNA expression libraries in 96 well-plates, served for the screening for recombinant adenoviruses, which contain the cDNAs for hAAT or hFIX. For the ELISAs, 96 well-plates were initially coated with commercial antibodies which bind hAAT (Anti-hAAT from the goat) and hFIX (Anti-hFIX from the mouse). Then the plates were incubated with 1:4 dilution of the cell culture supernatants to be tested. Antigen bound to the plates was then detected after incubation with POD-coupled antibodies (sheep-anti-hAAT-POD and rabbit-anti-hFIX-IgG followed by goat-anti-rabbit-IgG-POD) and addition of OPD by measurement of the absorption at 490 nm. Supernatants from non-infected cells, as well as supernatants of cells which had been infected with the “empty” donor virus AdlantisI, were used as negative controls.

For the isolation of recombinant adenoviruses, which contain the cDNAs for hAAT or hFIX, procedure was according to the schematic summarized in the FIGS. 27-29. In the first screening round (FIG. 27), 293 cells in 96 well-plates were infected with oligoclonal subpopulations of the purified adenoviral expression libraries, and the cells were lysed after 7 days in the plates by freeze/thaw lysis (masterplates S1A1=screening round 1 amplification round 1). For the amplification of the viruses with the objective of clearer signals in the ELISA, the supernatants of the masterplates S1A1 were used for the infection of a further 96 well-plates with 293 cells. After the cells were completely infected, they were in turn lysed through freeze/thaw lysis (masterplates S1A2=screening round 1 amplification round 2). The supernatants of the masterplates S1A2 were then tested for hAAT or hFIX by means of ELISA. Thereby, as a result of the first screening round, oligoclonal subpopulations could be identified in the masterplates S1A2, which contain recombinant adenoviruses with the hAAT cDNA or hFIX cDNA.

In the second screening round (FIG. 28) it was a matter then of reducing the complexity of these subpopulations. For this, the total titer was determined, first of all, of infectious particles in the corresponding wells of the masterplates S1A2. Then 293 cells in 96-well-plates were infected with a defined number of infectious particles from the positive wells of the masterplates S1A2 and the cells were lysed after 7 days in the plates through freeze/thaw lysis (masterplates S2A1=screening round 2 amplification round 1). For the amplification of the viruses with the objective of clearer signals in the ELISA, the supernatants of the masterplates A1S1 were then used again for the infection of a further 96 well plate with 293 cells. After the cells were completely infected, they were in turn lysed through freeze/thaw lysis (masterplates S2A2=screening round 2 amplification round 2). The supernatants of the masterplates S1A2 were then either directly tested for hAAT or hFIX by means of ELISA, or further used for the infection of a further 96 well-plates with 293 cells. Through this, the masterplates S2A3 were obtained, which were then tested for hAAT or hFIX by means of ELISA.

Thereby, as a result of the second screening round, low complexity subpopulations could be identified in the masterplates S2A2 and/or S2A3, which contain recombinant adenoviruses with the hAAT cDNA or hFIX cDNA. Their separation in clonal form can then be done according to the schematic in FIG. 29: By plaque assay on 293 cells, individual adenovirus clones can be obtained from the positive wells of the masterplates S2A2 and S2A3. These can then be amplified individually on 293 cells. Through testing of the cell culture supernatants by means of ELISA, the adenovirus clone can then be identified which contain the cDNA's for hAAT and hFIX.

Due to the high expression level of the hAAT gene in the liver, it was assumed that about 1/100 to 1/1000 of the viruses in the adenoviral expression libraries contain the hAAT cDNA. It thus appeared sufficient to employ in the first screening round, according to FIG. 27, three 96 well-plates each, in which for S1A1 50 infectious particles per well from AdlantisLIVERcDNAI and AdlantisLIVERcDNAII were used. With a total of 216 wells, this corresponds to 10,800 independent adenovirus clones each in the first screening round. The results of the hAAT ELISAs with the supernatants of the 3 masterplates each from S1A2 are shown in FIG. 30. With employment of AdlantisLIVERcDNAI, a total of 52 wells of the masterplates S1A2 were positive. With employment of AdlantisLIVERcDNAII, there were 65 positive wells in total. This corresponds to a frequency of 1/207 (AdlantisLIVERcDNAI) and 1/166 (AdlantisLIVERcDNAII) adenovirus clones, which agrees well with the frequency initially assumed (see above). Four positive wells of the masterplates S1A2 of the first screening round of AdlantisLIVERcDNAI were then selected and the titer of infectious particles was determined (masterplate S1A2 a well B9: ˜3×10⁸ IP/ml; masterplate S1A2 a well D1: ˜10⁸ IP/ml; masterplate S1A2 b well D10: ˜10⁸ IP/ml; masterplate S1A2 c well B8: ˜3×10⁸ IP/ml). In the second screening round, for the reduction of the complexity of these positive subpopulations, according to the schematic in FIG. 28, one 96 well-plate each was then infected with an infectious particle per well in S2A1 (total 72 wells=72 adenovirus clones, which covers the complexity of 50 independent adenovirus clones per well used in the first screening round). The results of the ELISAs with the supernatants of the four masterplates S2A3 are displayed in FIG. 31. In each case 2-4 wells per masterplate were positive, which indicates a successful isolation of recombinant adenoviruses which contain the hAAT cDNA.

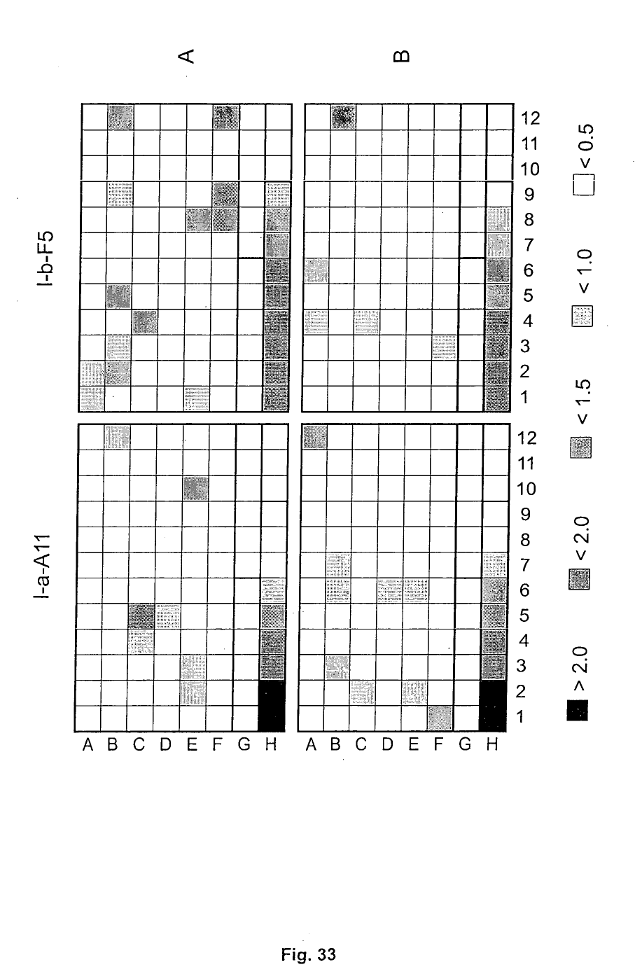

The screening for recombinant adenoviruses which contain the hFIX cDNA was carried out with AdlantisLIVERcDNAI only. Due to the low expression level of the hFIX-gene in the liver, it was assumed that less than 1/10,000 of the viruses in the adenoviral expression library contain the hFIX cDNA. In the first screening round, according to FIG. 27, nine 96-well-plates were used, in which 500 infectious particles per well from AdlantisLIVERcDNAI were used for S1A1. With a total of 648 wells, this corresponds to 324,000 independent adenovirus clones in the first screening round. The results of the hFIX ELISAs with the supernatants of the nine masterplates S1A2 are shown in FIG. 32. Only two wells were positive, which corresponds to a frequency of 1/162,000 adenovirus clones in the adenoviral expression library and in turn agrees well with the frequency initially assumed (see above). For both positive wells of the masterplates S1A2 of the first screening round, the titer of infectious particles was determined (masterplate S1A2 a well A11: ˜10⁸ IP/ml; masterplate S1A2 b well F5: ˜3×10⁸ IP/ml). In the second screening round, for the reduction of the complexity of these positive subpopulations according to the schematic in FIG. 28, one 96 well-plate each was then infected with ten infectious particles per well in S2A1 (total 144 wells=1440 adenovirus clones, which covers the complexity of 500 independent adenovirus clones per well used in the first screening round). The results of the ELISAs with the supernatants of the four masterplates S2A2 are shown in FIG. 33. In each case, 7-13 wells per masterplate were positive, which indicates a successful isolation of less complex subpopulations, which contain the hFIX cDNA.

For the generation of monoclonal subpopulations from the positive wells of the second screening round, individual virus plaques can be isolated by plaque assay on 293 cells, according to FIG. 29. The plaque isolates can then be amplified individually on 293 cells and the cell culture supernatants can be tested for hAAT or hFIX for verification by ELISA. Following this, the presence of the hAAT cDNA and hFIX cDNA in the adenovirus clones can be confirmed by sequencing.

By application of the invention-related system for the generation of recombinant adenoviruses, starting from mRNA, adenoviral cDNA expression libraries can thus be generated, which correspond to the general criteria for cDNA expression libraries: a complexity of about 10⁶ independent clones, a high content of complete cDNA's (>50%), and the presence also of cDNAs of genes expressed at low levels. Furthermore, it was shown that a screening of adenoviral cDNA expression libraries generated this way using masterplates with low complex subpopulations is suitable for the isolation of adenovirus clones with the required properties. Thus the invention-related system for the generation of the adenoviral cDNA expression libraries, as well as the invention-related methods for their screening, appear generally suitable to identify genes, which cause a detectable phenotype in a biological test system.

The invention thus concerns a novel system for the generation of recombinant adenoviruses (rAd); areas of application are, in particular, medicine, veterinary science, biotech, gene technology and the functional genome analysis.

A novel system for the generation of rAd is the content of the invention. The rAd are generated by site-specific insertion of foreign DNA into an infectious replicating virus. With this new system, clonal rAd populations can be generated faster and more simply as compared to previous methods. Furthermore, in contrast to previous methods, the new process enables the generation of complex mixed rAd populations. The content of the invention is furthermore the use of the new method of rAd generation for the construction of complex gene libraries in the adenoviral context, for example of cDNA expression libraries.

The rAd obtained in this way are usable for the transfer and the expression of genes in cells, as well as for the transfer of genetic material in animals and humans, with the objective of a gene therapy and/or vaccination. Furthermore, the complex rAd population obtained in this way (gene libraries) are usable for the isolation of new genes, as well as for the functional change or optimization of known genes.

The invention-related system for the rAd generation preferably consists of the following:
A donor virus, whose packaging signal (i) is partially deleted and (ii) is framed by parallel-oriented recognition sites for a site-specific recombinase,
A packaging cell line, which expresses the site-specific recombinase
Donor plasmids, which contain (i) one or two recognition sites for the site-specific recombinase, (ii) the complete viral packaging signal, (iii) where appropriate, two recognition sites for a rare cutting restriction endonuclease and (iv) insertion points for foreign DNA or inserted foreign DNA.

1. System for the generation of recombinant adenoviruses, comprising
(a) a donor virus with a partially deleted viral packaging signal, which is framed by two recognition sites for a site-specific recombinase,
(b) a packaging cell line, which expresses the site-specific recombinase and
(c) a donor plasmid, which contains one or two recognition sites for the site-specific recombinase, the complete viral packaging signal and insertion sites for foreign DNA and/or inserted foreign DNA.

2. System according to claim 1, wherein
it is suitable for the generation of a clonal population of recombinant adenoviruses, by employment of a clonal population of the donor plasmid.

3. System according to claim 1, wherein
it is suitable for the generation of a complex population of recombinant adenoviruses, by employment of a complex population of the donor plasmid.

4. System according to claim 1, wherein
a donor virus is used, which is derived from human adenoviruses.

5. System according to claim 1, wherein
a donor virus is used, which is derived from non-human adenoviruses.

6. System according to claim 1, wherein
in the donor virus at least one non-essential viral gene is deleted.

7. System according to claim 1, wherein
in the donor virus, at least one essential viral gene is deleted.

8. System according to claim 1, wherein
the rescue and propagation of the donor virus is done in a producer cell line, which makes available the deleted essential viral gene(s).

9. System according to claim 1, wherein
a donor virus is used, which is derived from the human adenovirus serotype 5 and contains a deletion of the essential E1-Region.

10. System according to claim 1, wherein
donor viruses derived from the human adenovirus serotype 5 with a deletion of the non-essential E3-region are used.

11. System according to claim 1, wherein
in the donor virus, there are two recognition sites for a site-specific recombinase of the Int family.

12. System according to claim 1, wherein
in the donor virus, there are two recognition sites for the Cre-recombinase.

13. A recombinant virus derived from the human adenovirus serotype 5, where it contains
(a) a deletion of the E1-Region,
(b) a deletion of the E3 region and
(c) a partially deleted viral packaging signal that
(d) is framed by parallel-oriented recognition sites for the Cre-recombinase.

14. A recombinant virus according to claim 13, wherein
the partially deleted viral packaging signal contains the A repeats I-V.

15. A recombinant virus according to claim 13, wherein
the partially deleted viral packaging signal contains the A repeats I, II, VI and VII.

16. System according to claim 1, wherein
a donor plasmid is used, which contains
(a) a bacterial replication origin,
(b) a bacterial resistance gene,
(c) a recognition site for the site-specific recombinase,
(d) a complete viral packaging signal, as well as
(e) an insertion site for foreign DNA and/or foreign DNA

17. System according to claim 1, wherein
a donor plasmid is used, which contains
(a) a bacterial replication origin,
(b) a bacterial resistance gene,
(c) an recognition site for the site-specific recombinase,
(d) a viral ITR,
(e) a complete viral packaging signal,
(f) an insertion site for foreign DNA and/or foreign DNA and
(g) two recognition sites for a rare cutting restriction endonuclease.

18. System according to claim 1, wherein
a donor plasmid is used, which contains
(a) a bacterial replication origin,
(b) a bacterial resistance gene,
(c) two recognition sites for the site-specific recombinase,
(d) a complete viral packaging signal, as well as
(e) an insertion site for foreign DNA and/or foreign DNA.

19. System according to claim 1, wherein
in the donor plasmid there is present the complete packaging signal of adenovirus for serotype 5.

20. System according to claim 1, wherein
in the donor plasmid there are present one or two recognition sites for a site-specific recombinase of the Int Family.

21. System according to claim 20, wherein
in the donor plasmid there are present one or two recognition sites for the Cre-recombinase.

22. System according to claim 1, wherein
in the donor plasmid, recognition sites are present for a rare cutting restriction endonuclease, with a recognition sequence more than 8 bp long.

23. System according to claim 22, wherein
in the donor plasmid there are present recognition sites for the rare cutting restriction endonuclease I-SceI.

24. System according to claim 1, wherein
in the donor plasmid there is present the 5′ITR of adenovirus serotype 5.

25. Donor plasmids for employment in a system according to claim 1.

26. System according to claim 1, wherein
the packaging cell line expresses a site-specific recombinase of the Int Family.

27. System according to claim 1, wherein
the packaging cell line, besides the site-specific recombinase, makes available essential viral gene(s) deleted, where appropriate, in the donor virus.

28. System according to claim 1, wherein
the packaging cell line expresses the Cre-recombinase and makes available the E1 gene products of adenovirus serotype 5.

29. System according to claim 1, wherein
the cell line CIN 1004 is used as a packaging cell line.

30. Use of the cell line CIN1004 for the generation of clonal or complex populations of recombinant adenoviruses.

31. System according to claim 1, wherein
a clonal population of the donor plasmid is used, with which an expression cassette is present as foreign DNA, which contains
(a) a promoter,
(b) the open reading frame of a gene,
(c) a polyadenylation signal,
(d) where appropriate, at least one insulator,
(e) where appropriate, at least one intron and
(f) where appropriate, at least one enhancer.

32. System according to claim 1, wherein
a complex population of the donor plasmid is used, with which there is present a mixture of different DNA sequences as foreign DNA.

33. System according to claim 32, wherein
there is present a mixture of non-coding DNA sequences as foreign DNA.

34. System according to claim 32, wherein
there is present a mixture of coding DNA sequences as foreign DNA.

35. System according to claim 32, wherein
there is present a mixture of expression units as foreign DNA, with which there are differently coding DNA sequences under the control of the same promoter and polyadenylation signal.

36. System according to claim 32, wherein
there is a cDNA library present as a mixture of coding sequences.

37. System according to claim 32, wherein
as a mixture of coding sequences, there is present a mixture of variants of an individual gene, which are distinguished in individual base pair positions at least, and/or contain insertions or deletions of at least one base pair.

38. System according to claim 32, wherein
there is present a mixture of expression units as foreign DNA, with which different promoters, which are distinguished in one bp position at least, and/or contain insertions or deletions of at least one base pair, which control the expression of the same coding DNA sequence.

39. Process for the generation of recombinant adenoviruses, comprising the steps
(a) Provision of a donor virus with an at least partially deleted viral packaging signal, which is framed by two recognition sites for a site-specific recombinase,
(b) Infection of a packaging cell line, which expresses the site-specific recombinase with the donor virus,
(c) Formation of a donor virus acceptor substrate through action of the site-specific recombinase on the donor virus,
(d) Transfection of donor plasmids, which contain one or two recognition sites for the site-specific recombinase, the complete viral packaging signal and insertion sites for foreign DNA and/or inserted foreign DNA into the donor virus infected packaging cell line and
(e) Formation of recombinant adenoviruses through action of the site-specific recombinase.

40. Process according to claim 39 for the generation of a clonal population of recombinant adenoviruses.

41. Process according to claim 39 for the generation of a complex population of recombinant adenoviruses.

42. Process according to claim 39, further comprising the step
(f) amplification of the recombinant adenoviruses.

43. Process according to claim 42, wherein
the amplification is done on cells which express the site-specific recombinase.

44. Process according to claim 42, wherein
the amplification is done on cells which express the site-specific recombinase.

45. Process according to claim 39, further comprising the step
(g) Purification of the recombinant adenoviruses through density gradient centrifugation or affinity chromatography.

46. Recombinant adenoviruses population produced using a process according to claim 39.

47. Recombinant adenovirus population according to claim 46, wherein
the recombinant population is a clonal population.

48. Recombinant adenovirus population according to claim 46, wherein
the recombinant population is a complex population.

49. Clonal adenovirus population according to claim 47, wherein
there is present an expression cassette as foreign DNA, which contains
(a) a promoter,
(b) an open reading frame of a gene,
(c) a polyadenylation signal,
(d) where appropriate, at least one insulator,
(e) where appropriate, at least one intron and,
(f) where appropriate, at least one enhancer.

50. Complex adenovirus population according to claim 48, wherein
there is present a mixture of different DNA sequences as foreign DNA.

51. Complex adenovirus population according to claim 48, wherein
there is present a mixture of non-coding DNA sequences as foreign DNA.

52. Complex adenovirus population according to claim 48, wherein
there is present a mixture of coding DNA sequences as foreign DNA.

53. Complex adenovirus population according to claim 48, wherein
there is present a mixture of expression units as foreign DNA, with which there are different coding DNA sequences under the control of the same promoter and polyadenylation signal.

54. Complex adenovirus population according to claim 48, wherein
there is present a cDNA library as a mixture of coding sequences.

55. Complex adenovirus population according to claim 48, wherein
there is present a mixture of variants of an individual gene as a mixture of coding sequences, which are distinguished at least in individual base pair positions, and/or insertions or deletions of at least one base pair.

56. Complex adenovirus population according to claim 48, wherein
there is present a mixture of expression units as foreign DNA, with which different promoters, which differ in one bp position at least, and/or contain insertions or deletions of at least one base pair, control the expression of the same coding DNA sequence.

57. Utilization of a recombinant adenovirus population, according to claim 46, for the transfer of genetic material in cells or/and animals, in particular into human cells or/and humans.

58. Utilization according to claim 57 for the gene transfer and the expression of genes in cells.

59. Utilization according to claim 57 for the transfer of genetic material into animals or/and humans for gene therapy or/and vaccination.

60. Utilization according to claim 57 for the gene transfer into cells or cell complexes, which exhibit changed, in particular, sick appearances.

61. Utilization according to claim 60 for the therapy of inherited, acquired or malignant disease.

62. Utilization according to claim 57 for the DNA vaccination, in particular for vaccination against pathogens, such as viruses, bacteria, as well as single-cell or multiple-cell eukaryotes, or for the vaccination against malignant or non-malignant cells and/or cell populations.

63. Utilization of a complex population of recombinant adenoviruses according to claim 53, for the isolation, where appropriate, of new genes which cause a certain phenotype in a cell-based test system.

64. Utilization of a complex population of recombinant adenoviruses according to claim 55, for the isolation of variants of a gene with changed properties.

65. Utilization of a complex population of recombinant adenoviruses according to claim 56, for the isolation of variants of a promoter with changed properties.

66. Utilization of a complex population of recombinant adenoviruses according to claim 51, for the isolation of sequences with certain binding sites for proteins.

67. Process for the generation of masterplates with clonal or low complexity sub-populations, from a complex population of adenoviruses, comprising
(a) the titration of the complex population of recombinant adenoviruses,
(b) the infection of producer cells cultivated in multititer plates, with only one or few infectious particles of the recombinant adenovirus population per multititer plate well,
(c) the lysis of the producer cells in the multititer plate after occurrence of the cytopathic effect and
(d) the storage of the masterplates in frozen status.

68. Masterplates available with a process according to claim 67.

69. Process for the identification of clonal or low complexity sub-populations from a complex population of adenoviruses, which cause a certain verifiable phenotype in a cell-based test system, comprising
(a) the utilization of the virus-containing supernatants of the lysed cells in masterplates, which are available in accordance with a process according to claim 67, for the infection of the cells of the functional test system,
(b) the implementation of the functional test with the infected cells of the test system and
(c) the identification of the well(s) of the masterplates, which contains/contain the viruses with the required functional properties.

70. Process according to claim 69, further comprising
(d) the clonal separation of the recombinant adenoviruses through plaque assay on a producer cell line,
(e) the cultivation of the thus obtained clonal recombinant adenovirus population and the characterization of the foreign DNA contained in it.

71-74. (canceled)

75. A method for isolating new genes which result in a certain phenotype in a cell-based test system comprising
(a) producing masterplates according to claim 67 for the infection of cells of a functional test system,
(b) implementing the functional test with the infected cells of the test system,
(c) identifying the well(s) of the masterplates, which contains/contain the viruses with the required functional properties,
(d) clonal separation of any recombinant adenoviruses through plaque assay on a producer cell line, and
(e) cultivating the thus obtained clonal recombinant adenovirus population and characterizing any foreign DNA contained in it in order to isolate new genes.

76. A method for isolating variants of a gene with changed properties, isolating variants of a promoter with changed properties or isolating sequences with certain binding sites for proteins, comprising
(a) producing masterplates according to claim 67 for the infection of cells of a functional test system,
(b) implementing the functional test with the infected cells of the test system,
(c) identifying the well(s) of the masterplates, which contains/contain the viruses with the required functional properties,
(d) clonally separating any recombinant adenoviruses through plaque assay on a producer cell line, and
(e) cultivating the thus obtained clonal recombinant adenovirus population and characterizing any foreign DNA contained in it in order to identify variants of a gene with changed properties, isolate variants of a promoter with changed properties and/or isolate sequences with certain binding sites for proteins.
